(19) United States
(12) Patent Application Publication
(10) Pub. No.: US 2026/0187763 A1
(43) Pub. Date: Jul. 2, 2026

(54) METHOD OF DETERMINING CONTRAST SCANNING PROTOCOL AND COMPUTER DEVICE

(71) Applicant: Shanghai United Imaging Healthcare Co., Ltd., Shanghai (CN)

(72) Inventors: Cuili Xue, Shanghai (CN); Gang Yin, Shanghai (CN); Yifeng Jiang, Shanghai (CN); Yangyang Lin, Shanghai (CN)

(21) Appl. No.: 19/437,189

(22) Filed: Dec. 30, 2025

(30) Foreign Application Priority Data
Dec. 30, 2024 (CN) 202411976614.3
Dec. 30, 2024 (CN) 202411976659.0

(51) Int. Cl. G06T 5/60; A61B 6/00; G06T 7/00; G16H 30/40

(52) U.S. Cl. CPC G06T 5/60; G06T 7/0012; G16H 30/40; A61B 6/481; A61B 6/545; G06T 2207/20081; G06T 2207/20084; G06T 2207/20192; G06T 2207/30096; G06T 2207/30168

(57) ABSTRACT

A method of determining a contrast scanning protocol and a computer device are provided. The method includes: obtaining a target scanned object physiological model of a target scanned object, inputting the target scanned object physiological model and a plurality of candidate contrast scanning protocols into a trained contrast simulation model to obtain enhancement effect parameters of the plurality of candidate contrast scanning protocols, and determining a recommended contrast scanning protocol from the plurality of candidate contrast scanning protocols according to the enhancement effect parameters of the plurality of candidate contrast scanning protocols.

### CROSS-REFERENCE TO RELATED APPLICATIONS

This application claims priority to Chinese Patent Application No. 202411976614.3, filed on Dec. 30, 2024, and Chinese Patent Application No. 202411976659.0, filed on Dec. 30, 2024, the contents of which are hereby incorporated by reference in their entireties.

### TECHNICAL FIELD

The present disclosure relates to the field of medical contrast imaging technology, and in particular, to a method of determining a contrast scanning protocol and a computer device.

### BACKGROUND

An enhanced scanning, achieved by intravenous injection of a contrast agent, can improve a contrast between tissues and organs, thereby more clearly displaying a relationship between lesions and surrounding structures as well as a size, a shape, and an extent of the lesions. For example, the enhanced scanning can be used for contrast-enhanced imaging of blood vessels, organs, and tissues. Currently, the formulation of contrast agent injection and scanning parameters largely relies on the experience of clinical physicians. The accuracy may be affected by expertise and experience of the physicians, and unreasonable contrast agent injection parameters may lead to suboptimal image enhancement effect or artifacts. It can be seen that the related art cannot efficiently and accurately acquire a contrast protocol tailored to an object to be scanned.

### SUMMARY

According to various embodiments of the present disclosure, a method of determining a contrast scanning protocol and a computer device are provided.

In a first aspect, a method of determining a contrast scanning protocol is provided, including: obtaining a target scanned object physiological model of a target scanned object, inputting the target scanned object physiological model and a plurality of candidate contrast scanning protocols into a trained contrast simulation model to obtain enhancement effect parameters of the plurality of candidate contrast scanning protocols, and determining a recommended contrast scanning protocol from the plurality of candidate contrast scanning protocols according to the enhancement effect parameters of the plurality of candidate contrast scanning protocols. The contrast simulation model is configured to simulate a flow characteristic of a contrast agent in the target scanned object and an enhancement condition of a target scanned region of the target scanned object under current scanning parameters, and output the enhancement effect parameters according to a result of the simulation.

In some embodiments, obtaining the target scanned object physiological model of the target scanned object further includes: obtaining target scanned region information and target injection region information of the target scanned object, obtaining a general physiological model according to the target scanned region information and the target injection region information, and adjusting, based on vital signs information of the target scanned object, the general physiological model to obtain the target scanned object physiological model corresponding to the target scanned object. The general physiological model characterizes physiological structural information between the target scanned region and the target injection region.

In some embodiments, adjusting, based on the vital signs information of the target scanned object, the general physiological model to obtain the target scanned object physiological model corresponding to the target scanned object further includes: determining the vital signs information by performing vital signs information extraction on the target scanned object, and adjusting the general physiological model according to the extracted vital signs information to obtain the target scanned object physiological model corresponding to the target scanned object. The vital signs information includes either or both of physiological parameters affecting changes in fluid flow characteristics in the target scanned region and medical information of the target scanned object.

In some embodiments, the plurality of candidate contrast scanning protocols are acquired by: determining a setting range of contrast injection parameters and a setting range of contrast scanning parameters, determining a plurality of injection parameters in the setting range of contrast injection parameters and a plurality of scanning parameters in the setting range of contrast scanning parameters, and obtaining the plurality of candidate contrast scanning protocols according to a combination result of the plurality of injection parameters and the plurality of scanning parameters.

In some embodiments, determining the recommended contrast scanning protocol from the plurality of candidate contrast scanning protocols according to the enhancement effect parameters of the plurality of candidate contrast scanning protocols further includes: obtaining a preset target enhancement effect parameter, determining similarities between the enhancement effect parameters of the candidate contrast scanning protocols and the preset target enhancement effect parameter, and designating a candidate contrast scanning protocol whose similarity satisfies a preset similarity condition as the recommended contrast scanning protocol.

In some embodiments, obtaining the preset target enhancement effect parameter further includes: determining the target enhancement effect parameter according to at least one of an imaging enhancement value, image quality evaluation information, or an image signal-to-noise ratio corresponding to a target medical image. The target medical image is a medical image that is expected to be obtained after performing contrast scanning based on a candidate contrast scanning protocol corresponding to the target medical image.

In some embodiments, the contrast simulation model is configured to simulate one or more of flow characteristics of a contrast agent, the one or more flow characteristics including: a time-varying distribution process of the contrast agent, a time for the contrast agent to reach the target scanned region, or a time for the contrast agent to exit the target scanned region.

In some embodiments, a training process of the contrast simulation model includes: determining a sample contrast scanning protocol configured for a sample scanned object to acquire an expected image enhancement effect, and determining, according to an actual enhancement effect obtained when performing sample contrast scanning on the sample scanned object based on the sample contrast scanning protocol, first enhancement effect evaluation information that is taken as a label; inputting sample vital signs information of the sample scanned object, a sample enhancement effect parameter corresponding to the expected image enhancement effect, and the sample contrast scanning protocol into an original simulation model to obtain second enhancement effect evaluation information output by the original simulation model; and adjusting the original simulation model according to a difference between the second enhancement effect evaluation information and the first enhancement effect evaluation information until a training end condition is satisfied, to obtain the trained contrast simulation model.

In some embodiments, inputting the sample vital signs information of the sample scanned object, the sample enhancement effect parameter corresponding to the expected image enhancement effect, and the sample contrast scanning protocol into the original simulation model to obtain the second enhancement effect evaluation information output by the original simulation model, further includes: inputting the sample vital signs information of the sample scanned object, the sample enhancement effect parameter corresponding to the expected image enhancement effect, and the sample contrast scanning protocol into the original simulation model; and obtaining, by the original simulation model, sample vital signs information vectors corresponding to the sample scanned object, a sample enhancement effect vector corresponding to the sample enhancement effect parameter, and a contrast scanning protocol vector corresponding to the sample contrast scanning protocol, determining, by the original simulation model, a feature combination result according to the sample vital signs information vectors, and outputting, by the original simulation model, the second enhancement effect evaluation information according to the feature combination result, a sample enhancement effect feature corresponding to the sample enhancement effect vector, and a contrast scanning protocol feature corresponding to the contrast scanning protocol vector.

In some embodiments, the original simulation model includes an input layer, a processing layer, a hidden layer, and an output layer that are sequentially connected.

Obtaining, by the original simulation model, sample vital signs information vectors corresponding to the sample scanned object, the sample enhancement effect vector corresponding to the sample enhancement effect parameter, and the contrast scanning protocol vector corresponding to the sample contrast scanning protocol, further includes: obtaining, the input layer, sample vital signs information vectors corresponding to the sample scanned object, the sample enhancement effect vector corresponding to the sample enhancement effect parameter, and the contrast scanning protocol vector corresponding to the sample contrast scanning protocol.

Determining, by the original simulation model, the feature combination result according to the sample vital signs information vectors, and outputting, by the original simulation model, the second enhancement effect evaluation information according to the feature combination result, the sample enhancement effect feature corresponding to the sample enhancement effect vector, and the contrast scanning protocol feature corresponding to the contrast scanning protocol vector, further includes: inputting the sample vital signs information vectors, the sample enhancement effect vector, and the contrast scanning protocol vector into the processing layer for feature extraction, and obtaining the feature combination result corresponding to the sample vital signs information vectors, the sample enhancement effect feature corresponding to the sample enhancement effect vector, and the contrast scanning protocol feature corresponding to the contrast scanning protocol vector; and inputting the feature combination result, the sample enhancement effect feature, and the contrast scanning protocol feature into the hidden layer for feature fusion, inputting a feature fusion result into the output layer, and obtaining the second enhancement effect evaluation information output by the output layer.

In some embodiments, the feature combination result includes features acquired in at least one of the following manners: determining consecutive features according to sample vital signs information vectors of first-type sample vital signs information in a plurality of pieces of sample vital signs information; encoding sample vital signs information vectors of second-type sample vital signs information in the plurality of pieces of sample vital signs information to obtain a discrete feature according to an encoding result; performing cross-combination on sample vital signs information vectors of third-type sample vital signs information in the plurality of pieces of sample vital signs information to obtain a cross-combination feature according to a cross-combination result; or performing feature extraction on sample vital signs information vectors of fourth-type sample vital signs information in the plurality of pieces of sample vital signs information to obtain a deep feature.

In some embodiments, the method further includes: obtaining one or more optimized contrast scanning protocols according to protocol adjustment information entered by a user for the recommended contrast scanning protocol, and adjusting the trained contrast simulation model for updating according to differences between the one or more optimized contrast scanning protocols and the recommended contrast scanning protocol, to obtain an updated trained contrast simulation model that matches a protocol configuration habit of the user.

In some embodiments, each of the one or more optimized contrast scanning protocols is corresponding to a contrast purpose, and the contrast purpose indicates a medical task applied to a medical image obtained after performing the contrast scanning based on the optimized contrast scanning protocol. Adjusting the trained contrast simulation model for updating according to differences between the one or more optimized contrast scanning protocols and the recommended contrast scanning protocol, to obtain the updated trained contrast simulation model that matches the protocol configuration habit of the user, further includes: determining a plurality of optimized contrast scanning protocols corresponding to the same contrast purpose; and for each contrast purpose, adjusting the trained contrast simulation model according to differences between the plurality of optimized contrast scanning protocols corresponding to the contrast purpose and a corresponding recommended contrast scanning protocol, to obtain the updated trained contrast simulation model that matches a protocol configuration habit of the user and the contrast purpose.

In a second aspect, a method of determining a contrast scanning protocol is provided, including: obtaining a candidate contrast scanning protocol of a target scanned object, a target enhancement effect parameter configured to indicate an expected image enhancement effect for a user, and vital signs information of the target scanned object; inputting the candidate contrast scanning protocol, the target enhancement effect parameter, and the vital signs information of the target scanned object into a trained contrast simulation model, to obtain enhancement effect evaluation information of the candidate contrast scanning protocol that is output by the trained contrast simulation model; and determining a recommended contrast scanning protocol according to the enhancement effect evaluation information. The enhancement effect evaluation information indicates a degree of difference between an image enhancement effect obtained based on the candidate contrast scanning protocol and the expected image enhancement effect.

In some embodiments, the contrast simulation model is obtained by supervised training based on a sample contrast scanning protocol with a label of enhancement effect evaluation information and vital signs information of a sample scanned object.

In some embodiments, a training process of the contrast simulation model includes: determining a sample contrast scanning protocol configured for a sample scanned object to acquire an expected image enhancement effect, and determining, according to an actual enhancement effect obtained when performing sample contrast scanning on the sample scanned object based on the sample contrast scanning protocol, first enhancement effect evaluation information that is taken as a label; inputting sample vital signs information of the sample scanned object, a sample enhancement effect parameter corresponding to the expected image enhancement effect, and the sample contrast scanning protocol into an original simulation model to obtain second enhancement effect evaluation information output by the original simulation model; and adjusting the original simulation model according to a difference between the second enhancement effect evaluation information and the first enhancement effect evaluation information until a training end condition is satisfied, to obtain a trained contrast simulation model.

In some embodiments, inputting the sample vital signs information of the sample scanned object, the sample enhancement effect parameter corresponding to the expected image enhancement effect, and the sample contrast scanning protocol into the original simulation model to obtain the second enhancement effect evaluation information output by the original simulation model, further includes: inputting the sample vital signs information of the sample scanned object, the sample enhancement effect parameter corresponding to the expected image enhancement effect, and the sample contrast scanning protocol into the original simulation model; and obtaining, by the original simulation model, sample vital signs information vectors corresponding to the sample scanned object, a sample enhancement effect vector corresponding to the sample enhancement effect parameter, and a contrast scanning protocol vector corresponding to the sample contrast scanning protocol, determining, by the original simulation model, a feature combination result according to the sample vital signs information vectors, and outputting, by the original simulation model, the second enhancement effect evaluation information according to the feature combination result, a sample enhancement effect feature corresponding to the sample enhancement effect vector, and a contrast scanning protocol feature corresponding to the contrast scanning protocol vector.

In some embodiments, the original simulation model includes an input layer, a processing layer, a hidden layer, and an output layer that are sequentially connected.

Obtaining, by the original simulation model, vital signs information vectors corresponding to the sample scanned object, the sample enhancement effect vector corresponding to the sample enhancement effect parameter, and the contrast scanning protocol vector corresponding to the sample contrast scanning protocol, further includes: obtaining, by the input layer, sample vital signs information vectors corresponding to the sample scanned object, the sample enhancement effect vector corresponding to the sample enhancement effect parameter, and the contrast scanning protocol vector corresponding to the sample contrast scanning protocol.

Determining, by the original simulation model, the feature combination result according to the sample vital signs information vectors, and outputting, by the original simulation model, the second enhancement effect evaluation information according to the feature combination result, the sample enhancement effect feature corresponding to the sample enhancement effect vector, and the contrast scanning protocol feature corresponding to the contrast scanning protocol vector, further includes: inputting the sample vital signs information vectors, the sample enhancement effect vector, and the contrast scanning protocol vector into the processing layer for feature extraction, and obtaining the feature combination result corresponding to the sample vital signs information vectors, the sample enhancement effect feature corresponding to the sample enhancement effect vector, and the contrast scanning protocol feature corresponding to the contrast scanning protocol vector; and inputting the feature combination result, the sample enhancement effect feature, and the contrast scanning protocol feature into the hidden layer for feature fusion, inputting a feature fusion result into the output layer, and obtaining the second enhancement effect evaluation information output by the output layer.

In some embodiments, the feature combination result includes features acquired in at least one of the following manners: determining consecutive features according to sample vital signs information vectors of first-type sample vital signs information in a plurality of pieces of sample vital signs information; encoding sample vital signs information vectors of second-type sample vital signs information in the plurality of pieces of sample vital signs information to obtain a discrete feature according to an encoding result; performing cross-combination on sample vital signs information vectors of third-type sample vital signs information in the plurality of pieces of sample vital signs information to obtain a cross-combination feature according to a cross-combination result; or performing feature extraction on sample vital signs information vectors of fourth-type sample vital signs information in the plurality of pieces of sample vital signs information to obtain a deep feature.

In some embodiments, the method further includes: obtaining one or more optimized contrast scanning protocols according to protocol adjustment information entered by a user for the recommended contrast scanning protocol, and adjusting the trained contrast simulation model for updating according to differences between the one or more optimized contrast scanning protocols and the recommended contrast scanning protocol, to obtain an updated trained contrast simulation model that matches a protocol configuration habit of the user.

In some embodiments, each of the optimized contrast scanning protocols is corresponding to a contrast purpose, which indicates a medical task applied to a medical image obtained after performing the contrast scanning based on the optimized contrast scanning protocol. Adjusting the contrast simulation model for updating according to differences between the optimized contrast scanning protocols and the recommended contrast scanning protocol, to obtain the contrast simulation model that matches the protocol configuration habit of the user, further includes: determining a plurality of optimized contrast scanning protocols corresponding to the same contrast purpose; and for each contrast purpose, adjusting the contrast simulation model according to differences between the plurality of optimized contrast scanning protocols corresponding to the contrast purpose and a corresponding recommended contrast scanning protocol, to obtain the contrast simulation model that matches the protocol configuration habit of the user and the contrast purpose.

In some embodiments, obtaining the candidate contrast scanning protocol of the target scanned object further includes: determining injection parameters and scanning parameters, and obtaining the candidate contrast scanning protocol according to the injection parameters and the scanning parameters.

In a third aspect, a computer device is further provided in the present disclosure, including a memory and a processor. The memory stores a computer program, and when the computer program is executed by the processor, steps of any one of the foregoing methods are implemented.

In a fourth aspect, a computer-readable storage medium is further provided in the present disclosure, storing a computer program. The computer program is executed by a processor to implement steps of any one of the foregoing methods.

In a fifth aspect, a computer program product is further provided in the present disclosure, storing a computer program. The computer program is executed by a processor to implement steps of any one of the foregoing methods.

Details of one or more embodiments of the present disclosure are set forth in the accompanying drawings and description below to make other features, objects, and advantages of the present disclosure clearer and more comprehensible.

### BRIEF DESCRIPTION OF THE DRAWINGS

To more clearly illustrate the technical solutions in the embodiments of the present disclosure, the drawings required for describing the embodiments will be briefly introduced below. It is obvious that the drawings in the following description are only some embodiments of the present disclosure. For those skilled in the art, other drawings may also be obtained from these disclosed drawings without creative efforts.

FIG. 1 is a flowchart of a method of determining a contrast scanning protocol in an embodiment.

FIG. 2 is a schematic diagram of an input parameter panel in an embodiment.

FIG. 3 is flowchart of steps of constructing a target scanned object physiological model in an embodiment.

FIG. 4 is a schematic diagram of an output parameter panel in an embodiment.

FIG. 5 is a schematic diagram of an enhanced contrast scanning system in an embodiment.

FIG. 6 is a flowchart of another method of determining a contrast scanning protocol in an embodiment.

FIG. 7 is a flowchart of a method of determining a contrast scanning protocol in an embodiment.

FIG. 8 is a flowchart of steps of constructing a contrast simulation model in an embodiment.

FIG. 9 is a flowchart of training a contrast simulation model in an embodiment.

FIG. 10 is a schematic diagram of a contrast simulation model in an embodiment.

FIG. 11 is a schematic diagram of feature processing in an embodiment.

FIG. 12 is a flowchart of optimizing a contrast simulation model in an embodiment.

FIG. 13 is a structural block diagram of an apparatus of determining a contrast scanning protocol in an embodiment.

FIG. 14 is a structural block diagram of an apparatus of determining a contrast scanning protocol in another embodiment.

FIG. 15 is an internal structural diagram of a computer device in an embodiment.

FIG. 16 is an internal structural diagram of another computer device in an embodiment.

### DETAILED DESCRIPTION

In order to make the objectives, technical solutions, and advantages of the present disclosure clearer, the present disclosure is further described in detail below with reference to the accompanying drawings and embodiments. It should be understood that the specific embodiments described herein are merely used to explain the present disclosure, and are not intended to limit the present disclosure.

To enable those skilled in the art to better understand the present disclosure, the related technologies will be first introduced below.

When scanning a target scanned object and obtaining corresponding medical images, if a density of a lesion tissue is close to that of a normal tissue, the lesion tissue may not be easily distinguishable in the medical images. In such cases, an enhanced scanning can be performed by injecting a contrast agent into the scanned object. After the introduction of the contrast agent, different tissue structures or different pathological conditions exhibit distinct characteristics and patterns in terms of the amount of the absorbed contrast agent (such as iodine content of tissues or organs) and the distribution of the absorbed contrast agent (such as iodine distribution in tissues or organs). This not only enhances the contrast of the medical images and clarifies boundaries between the lesion tissue and the normal tissue, but also makes the density, a shape, a size, and the like of the lesion tissue more prominent. Consequently, the contrast between the tissue and the organ can be improved, allowing for clearer visualization of a relationship between the lesion and surrounding structures, as well as the size, the shape, and an extent of the lesion. In practical application, this method can be used for contrast-enhanced imaging of blood vessels, organs, tissues, and the like.

The enhanced scanning, achieved by intravenous injection of a contrast agent, can improve the contrast between the tissue and the organ, thereby more clearly displaying the relationship between the lesion and the surrounding structures, as well as the size, the shape, and the extent of the lesion. For example, the enhanced scanning can be used for contrast-enhanced imaging of blood vessels, organs, and tissues. Currently, the formulation of contrast agent injection and scanning parameters largely relies on the experience of clinical physicians. The accuracy may be affected by expertise and experience of the physicians, and unreasonable contrast agent injection parameters may lead to suboptimal image enhancement effect or artifacts.

There are numerous individual factors affecting actual effect of a contrast protocol, and complex interaction may exist among these factors. Relying solely on fixed rules to set the contrast protocol may compromise the accuracy and effectiveness of the protocol, thereby reducing the adaptability between the ultimately acquired contrast protocol and the scanned object. Thus, a contrast solution well-adapted to the scanned object cannot be efficiently and accurately determined according to the related art.

On this basis, a method and an apparatus of determining a contrast scanning protocol, a computer device, a computer-readable storage medium, and a computer program product are provided in the present disclosure, so as to at least solve the above technical problem.

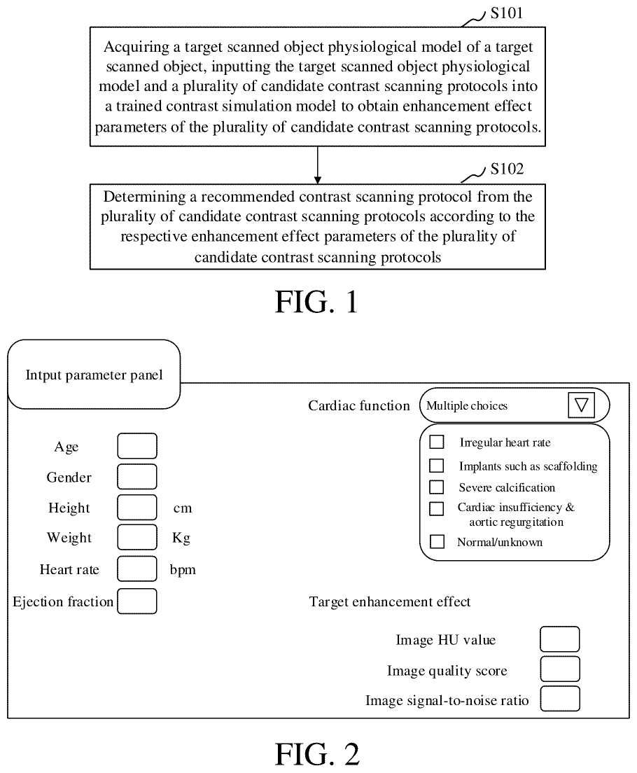

In an embodiment, referring to FIG. 1, a method of determining a contrast scanning protocol is provided. This embodiment is described by using an example in which the method is applied to a terminal device. In some examples, the terminal device may be a medical imaging device, such as a Computed Tomography (CT) device, a Magnetic Resonance Imaging (MRI) device, an Ultrasonic Contrast imaging device, or the like. It may be understood that the method may also be applied to a server, or may be applied to a system including a terminal and a server and implemented through interaction between the terminal and the server. In this embodiment, the method of determining the contrast scanning protocol includes the following step 101 and step 102.

Step 101 includes obtaining a target scanned object physiological model of a target scanned object, inputting the target scanned object physiological model and a plurality of candidate contrast scanning protocols into a trained contrast simulation model to obtain enhancement effect parameters of the plurality of candidate contrast scanning protocols. The contrast simulation model is configured to simulate a flow characteristic of a contrast agent in the target scanned object and an enhancement condition of a target scanned region of the target scanned object under current scanning parameters, and output the enhancement effect parameters according to a result of the simulation.

The target scanned object may be any object to be injected with a contrast agent for scanning, and the target scanned region may be a part or a region of the target scanned object for which corresponding medical images are to be acquired via contrast.

In specific implementation, a corresponding physiological model may be constructed for the target scanned object to obtain the target scanned object physiological model. The target scanned object physiological model may be a physiological model for modeling and simulating physiological processes of the target scanned object. The physiological model may describe and simulate functions and interactions of various tissues, organ structures, or systems in a body of the target scanned object through methods such as mathematical equations, statistical models, or computer simulations. For example, mathematical equations, formulas, and algorithms may be used to describe a blood flow process in the body of the target scanned subject. In the step, the target scanned object physiological model may be constructed for a specified target scanned object.

After the target scanned object physiological model is obtained, the target scanned object physiological model and the plurality of candidate contrast scanning protocols may be input into the trained contrast simulation model, to obtain the corresponding enhancement effect parameters output by the contrast simulation model for each of the plurality of candidate contrast scanning protocols.

The enhancement effect may indicate a degree of a difference between the lesion tissue and the normal tissue on an expected target medical image after the target scanned object is subjected to enhanced scanning by using the candidate contrast scanning protocol, and may also be understood as a degree of prominence of an imaging effect for the lesion tissue. A stronger enhancement effect can lead to clearer boundaries between the lesion tissue and the normal tissue and allow the display of the density, the shape, the size, and the like of the lesion tissue more prominent. In this embodiment, the enhancement effect of the expected target medical image may be specifically represented by the enhancement effect parameters. As indicators for quantitatively evaluating the effect of the enhanced scanning, the enhancement effect parameters may be quantitative information. For example, the degree of the difference between the lesion tissue and the normal tissue may be represented by specific image parameters. The enhancement effect parameters may also be qualitative information. For example, the degree of the prominence of the lesion tissue relative to the normal tissue may be represented by levels.

The contrast simulation model may receive input candidate contrast scanning protocols and the target scanned object physiological model, and then output the enhancement effect parameters expected when the candidate contrast scanning protocols are applied to the target scanned object. In some exemplary embodiments, the contrast simulation model may be obtained by training based on a neural network model. For example, a neural network model such as a deep neural network or a multi-layer perceptron may be trained through supervised training to obtain the contrast simulation model.

In related technologies, in some alternative implementations, the contrast protocols may be output by the constructed model. Specifically, a three-dimensional volumetric model may be constructed to model a region of interest (e.g., a scanned region) and perform fluid dynamics simulations to simulate the flow of the contrast agent in the region. However, this method requires pre-obtaining image data of a vascular system through imaging systems such as CT or MRI systems, and does not consider the influence of other vital signs information of the scanned object on fluid dynamics parameters of the region. When individual differences exist among scanned objects, it may lead to significant discrepancies between simulation results and the actual flow condition of the contrast agent.

In this embodiment, the target scanned object physiological model that characterizes systematic physiological processes of the target scanned object may be acquired. Then, the plurality of candidate contrast scanning protocols and the target scanned object physiological model may be input to the trained contrast simulation model. For the input target scanned object physiological model and each of the candidate contrast scanning protocols, the contrast simulation model can simulate the flow characteristics of the contrast agent in the target scanned object according to the candidate contrast scanning protocol and the target scanned object physiological model. In addition, the contrast simulation model can simulate enhancement conditions of the target scanned region of the target scanned object under the current scanning parameters of the candidate contrast scanning protocol. Thus, the enhancement effect parameters can be output by integrating simulation results from multiple aspects.

In some examples, the contrast simulation model may be configured to simulate one or more of the following flow characteristics of the contrast agent: a time-varying distribution process of the contrast agent, a time for the contrast agent to reach the target scanned region, and a time for the contrast agent to exit the target scanned region.

Step 102 includes determining a recommended contrast scanning protocol from the plurality of candidate contrast scanning protocols according to the enhancement effect parameters of the plurality of candidate contrast scanning protocols.

At this step, after the enhancement effect parameters of the plurality of candidate contrast scanning protocols are obtained, the enhancement effect parameters of the plurality of candidate contrast scanning protocols may be compared, and the recommended contrast scanning protocol may be determined from the plurality of candidate contrast scanning protocols according to a result of the comparison.

Specifically, since the protocol parameters contained in different candidate contrast scanning protocols vary, such as differences in injection parameters or scanning parameters, the final obtained enhancement effect parameters may also be different. Taking the plurality of candidate contrast scanning protocols including a first candidate contrast scanning protocol and a second candidate contrast scanning protocol as an example, the enhancement effect parameters predicted based on the first candidate contrast scanning protocol indicates that the boundaries between the lesion tissue and the normal tissue in the target medical image obtained based on the first candidate contrast scanning protocol would be the clearest. In contrast, the enhancement effect parameters predicted based on the second candidate contrast scanning protocol indicates that the boundaries between the lesion tissue and the normal tissue in the target medical image obtained based on the second candidate contrast scanning protocol would be blurry, with a small difference in image signals between the lesion tissue and the normal tissue. In this regard, by comparing the enhancement effect parameters of the plurality of candidate contrast scanning protocols, the candidate contrast scanning protocol capable of achieving a better enhancement effect can be selected from the plurality of candidate contrast scanning protocols as the recommended contrast scanning protocol.

In an embodiment, a user may set priorities for a plurality of enhancement effect parameters. For example, an imaging enhancement value may be set as having a first priority, an image quality score may be set as having a second priority, and an image signal-to-noise ratio may be set as having a third priority. Subsequently, for each of the plurality of candidate contrast scanning protocols, the trained contrast simulation model may output respective metric evaluation values corresponding to each of enhancement effect parameters. For example, three metric evaluation values of the imaging enhancement value, the image quality score, and the image signal-to-noise ratio for each of candidate contrast scanning protocols may be output. Then, the plurality of candidate contrast scanning protocols may be ranked according to the priorities of the enhancement effect parameters and specific metric evaluation values of the candidate contrast scanning protocols corresponding to the enhancement effect parameters, to determine the recommended contrast scanning protocol. Continuing with the example where the priorities for the imaging enhancement value, the image quality score, and the image signal-to-noise ratio are the first priority, the second priority, and the third priority, respectively: during ranking, a candidate contrast scanning protocol with the optimal imaging enhancement value may be determined as a first recommended contrast scanning protocol. When imaging enhancement values of the plurality of candidate contrast scanning protocols are the same, a candidate contrast scanning protocol with the optimal image quality score may be further determined as the first recommended contrast scanning protocol, and so on, until the recommended contrast scanning protocol is obtained.

In another embodiment, the recommended contrast scanning protocol may also be generated based on weighted values of the plurality of enhancement effect parameters. For example, for each of candidate contrast scanning protocols, a comprehensive score of each of the candidate contrast scanning protocols may be calculated according to specific metric evaluation values of the plurality of enhancement effect parameters for the protocol and respective weights of the plurality of enhancement effect parameters. Subsequently, the recommended contrast scanning protocol may be determined according to the comprehensive scores of respective candidate contrast scanning protocols.

According to the method of determining the contrast scanning protocol, the target scanned object physiological model of the target scanned object is acquired. The target scanned object physiological model and the plurality of candidate contrast scanning protocols are input into the trained contrast simulation model, and the enhancement effect parameters of the plurality of candidate contrast scanning protocols are obtained. The contrast simulation model is configured to simulate the flow characteristic of the contrast agent in the target scanned object and the enhancement condition of the target scanned region of the target scanned object under the current scanning parameters, and output the enhancement effect parameters according to the result of the simulation. Subsequently, the recommended contrast scanning protocol is determined from the plurality of candidate contrast scanning protocols according to the enhancement effect parameters of the plurality of candidate contrast scanning protocols. In this embodiment, the target scanned object physiological model can be used to fully describe physiological information of the target scanned object. By inputting the target scanned object physiological model and the candidate contrast scanning protocols into the contrast simulation model, it is possible to simulate the flow condition of the contrast agent and the enhancement condition of the target scanned region on the basis of considering various individual influencing factors of the target scanned object. This allows for accurate and effective prediction of the contrast effect when various candidate contrast scanning protocols are applied to the target scanned object. Consequently, when generating various candidate contrast agent protocols, an adaptive contrast protocol can be efficiently and personally provided for the target scanned object, and thus superior enhanced scanning effect can be achieved.

In an exemplary embodiment, obtaining the target scanned object physiological model of the target scanned object may include: determining target scanned region information and target injection region information of the target scanned object, obtaining a general physiological model from a preset model library according to the target scanned region information and the target injection region information, and adjusting the general physiological model to obtain the target scanned object physiological model corresponding to the target scanned object. The general physiological model may characterize physiological structural information between the target scanned region and the target injection region.

In specific implementation, the target scanned region information and target injection region information may be acquired. The target scanned region information may include information indicating a target scanned region on the target scanned object, and the target injection region information may include information indicating a target injection region for injecting of the contrast agent on the target scanned object. In some examples, the target scanned region information may include a specific scanned part, or may include a scanning protocol. For example, the target scanned region information may include a scanned region “chest” and a scanning protocol “coronary imaging”, and the target injection region information may include “right antecubital vein”.

After the target scanned region information and the target injection region information are determined, the corresponding general physiological model may be invoked from the preset model library according to the target scanned region information and the target injection region information.

Specifically, since physiological structures vary across different scanned regions, different scanning protocols may yield different imaging effects, and different injection regions may lead to varying drug distributions, respective general physiological models may be constructed in advance for combinations of at least two pieces of information of different scanned regions, scanning protocols, and injection regions. That is, the general physiological model may be constructed based on the injection region and at least one of the scanned region or the scanning protocol. This general physiological model may be understood as a physiological model obtained according to data statistics (such as statistics of a large amount of clinical data or experimental data) and stored in a model library. In some exemplary embodiments, the general physiological model may include one or more types of information about a blood vessel length, a blood vessel morphology, a blood flow velocity, and an organ model between the scanned region and the injection region, and may further include size information and/or fat content of the scanned region. Thus, after the target scanned region information and the target injection region information of the target scanned object are determined, the corresponding general physiological model can be invoked from the model library.

The general physiological model has universality, can broadly represent the common physiological process of multiple scanned objects, and has relatively low relevance or correspondence to a specific scanned object. However, the flow of the contrast agent within the body and the actual contrast effect may be influenced by various individual factors. If the general physiological model is directly input into the contrast simulation model to simulate the flow condition of the contrast agent and the enhancement condition of the target scanned region, the actual enhancement effect may deviate from expectations when the individual factors are ignored. In this regard, further adjustment can be performed on the basis of the general physiological model, and the target scanned object physiological model corresponding to the target scanned object can be obtained according to a result of the adjustment, so that the target scanned object physiological model can reflect individual characteristic information in a targeted and differentiated manner.

In this embodiment, on one hand, the target scanned object physiological model can be obtained by adjusting the general physiological model, avoiding regenerating the target scanned object physiological model each time and thereby improving the efficiency of obtaining the target scanned object physiological model. On the other hand, by obtaining the target scanned object physiological model corresponding to the target scanned object, the contrast simulation model can predict the simulation effect according to the personalized and diverse individual physiological factors of the target scanned object, which facilitates improving the prediction accuracy of the enhancement effect.

In an exemplary embodiment, adjusting the general physiological model to obtain the target scanned object physiological model corresponding to the target scanned object may include the following steps: performing vital signs information extraction on the target scanned object, and adjusting the general physiological model according to the extracted vital signs information to obtain the target scanned object physiological model corresponding to the target scanned object. The vital signs information may include either or both of physiological parameters affecting changes in fluid flow characteristics in the target scanned region, and medical information of the target scanned object.

In practical applications, various types of vital signs information of the target scanned object may be acquired. In some embodiments, the flow characteristics of the contrast agent in the target scanned region may be affected by fluid dynamics parameters of the region in the target scanned region, which may be related to the physiological parameters of the target scanned object. Therefore, the extracted vital signs information may include individual physiological parameters that affect the changes of the fluid flow characteristics in the target scanned region, such as one or more of heart rate, blood pressure, cardiac function, and ejection fraction. The extracted vital signs information may also include other physiological parameters, enabling the subsequently obtained target scanned object physiological model to more comprehensively and accurately characterize the physiological characteristics of the target scanned object. For example, the physiological parameter may further include one or more physiological parameters such as an age, a gender, a height, a weight, a lean body weight, and a body surface area.

On the other hand, the flow and actual contrast effect of the contrast agent on the target scanned object may also be affected by the own abnormal conditions of the target scanned object. For example, a target scanned object with cardiac dysfunction may experience uneven distribution or overly rapid metabolism of the contrast agent in the body. Similarly, arteriosclerosis may cause thickening of vascular walls and narrowing of the lumen, thereby affecting the flow rate and distribution of the contrast agent. Therefore, the medical information of the target scanned object may also be acquired as part of the vital signs information. In some examples, the medical information may include one or more of diagnosis conclusions, disease information, treatment records, and CT scan images.

In some alternative embodiments, the vital signs information extracting result corresponding to the target scanned object may be acquired through a vital signs monitoring system deployed in a scanning room. For example, individual physiological parameters of the target scanned object may be detected by using one or more of a camera, a pressure sensor, and a wearable device arranged in the scanning room in combination with an intelligent recognition algorithm, to obtain a height parameter and a weight parameter of the target scanned object. In addition, scan-related medical information may also be extracted based on the medical records of the target scanned object. For example, parameters such as an age, a gender, a height, and a weight of the target scanned object may be acquired from an information system, and information related to the scanned region such as lesion details and ejection fraction may be automatically extracted from the records of medical information. In some other embodiments, a control interface as shown in FIG. 2 may also be provided to acquire information such as cardiac function status and disease records by inquiring with the target scanned object, allowing manual input of the vital signs information of the target scanned subject into a device. It should be emphasized that various vital signs information of the target scanned object obtained in the present disclosure is acquired after sufficient authorization from the target scanned object is obtained.

According to different scanned regions and scanning protocols, the required vital signs parameters may also be different. In some embodiments, the vital signs information extraction may be performed on the target scanned object according to the target scanned region information to obtain vital signs information affecting the fluid flow characteristics in the target scanned region.

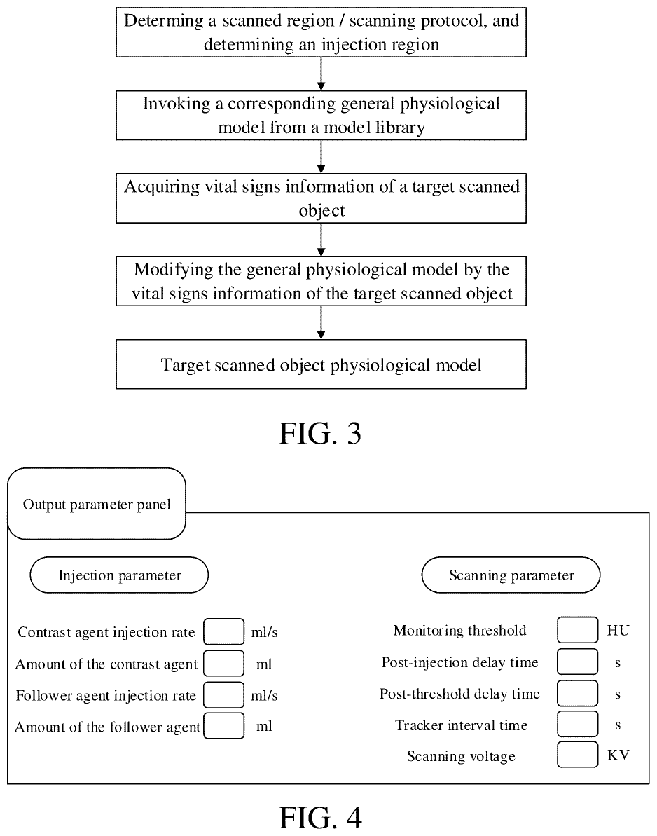

Subsequently, referring to FIG. 3, parameter correction may be performed on the general physiological model by using an extracting result of the vital signs information, to obtain the target scanned object physiological model. For example, if the scanned region is a chest and the scanning protocol is coronary scanning, cardiac function-related records may be extracted from the medical information of the target scanned object. Alternatively, a current scanned calcification score image may be used, or the cardiac function status may be manually entered after inquiry by the technician, to obtain the extracting result of the vital signs information including key vital signs information. The blood flow velocity in the scanned region in the general physiological model may be affected by factors such as an ejection fraction and a heart rate. For example, the general physiological model may include R(x), which may be expressed as follows:

R(x)=C(x, Ef)+Q(x, Hr)+ . . .

In the above formula, R(x) represents the blood flow velocities corresponding to different vascular positions between the injection region and the scanned region, C(x, EF) represents a function related to the vascular position and the ejection fraction, and Q(x, HR) represents a function of the vascular position and the heart rate. After obtaining the extracting result of the vital signs information, the blood flow velocity in the general physiological model may be adjusted based on the actual ejection fraction and the heart rate of the target scanned object included in the extracting result of the vital signs information, to obtain the target scanned object physiological model.

In some possible implementations, obtaining the contrast protocol may also involve obtaining individual information of the scanned object. For example, the required contrast agent dose for an imaging examination may be calculated according to some CT scanning parameters and the weight of the scanned object in combination with fixed formulas. Alternatively, reference indicators for contrast agent injection may be determined according to a lean body mass, and adjusted based on the age. However, the method for calculating the contrast agent dose and determining the contrast protocol based on the formula mainly refers to indicators such as weight, height, body fat rate, or lean body mass. In addition, the age and the gender may also be considered for coefficient adjustment in some methods. Nevertheless, the contrast protocol obtained through these methods may fail to effectively adapt to the scanned object, resulting in suboptimal contrast effects ultimately obtained. In contrast, in this embodiment, by obtaining the vital signs information of the target scanned object and adjusting the general physiological model based on the vital signs information, various individual factors affecting the flow condition of the contrast agent in the body can be fully considered, such as the heart rate, the cardiac function, the ejection fraction, the disease history and the like of the target scanned object, and thus the simulation accuracy of the contrast simulation model can be effectively improved.

In an exemplary embodiment, the plurality of candidate contrast scanning protocols may be acquired by the following steps: determining a setting range of contrast injection parameters and a setting range of contrast scanning parameters, determining a plurality of injection parameters in the setting range of contrast injection parameters and a plurality of scanning parameters in the setting range of contrast scanning parameters, and obtaining the plurality of candidate contrast scanning protocols according to a combination result of the plurality of injection parameters and the plurality of scanning parameters.

In practical applications, the setting range of contrast injection parameters and the setting range of contrast scanning parameters may be set by the user according to actual contrast scanning requirements.

The setting range of contrast injection parameters may be used to limit a variation range of the injection parameters. The injection parameters may include one or more parameters related to a drug injection in the contrast process, and a manner for the drug injection in the contrast process may be controlled by the injection parameters. In some exemplary embodiments, the injection parameters may include one or more of a contrast agent injection rate, a contrast agent injection duration, a contrast agent injection volume, a follower agent identifier, a follower agent type, a follower agent injection rate, a follower agent injection duration, and a follower agent injection volume.

The setting range of contrast scanning parameters may be configured to limit a variation range of the scanning parameters. The scanning parameters may include one or more parameters configured to control a scanning mode of a medical imaging device in the contrast process, and the scanning mode for the target scanned object in the contrast process may be controlled by the scanning parameters. In some exemplary embodiments, the scanning parameters may include one or more of the following parameters related to the enhancement effect: a tube voltage and a tube current. In addition, the scanning parameters may also include parameters such as a monitoring threshold and a scanning delay value. In some exemplary embodiments, several related scanning parameters may be provided to the user according to a scanning device selected by the user, and then the setting range of contrast scanning parameters input by the user for each scanning parameter may be acquired. For example, when the scanning is triggered by means of Bolus-Tracking after injection of the contrast agent, recommended scanning parameter may include some or all of parameters including the monitoring threshold, a post-injection delay time, a post-threshold delay time, a Tracker interval time, and a scanning voltage.

After the setting range of contrast injection parameters and the setting range of contrast scanning parameters are obtained, the plurality of injection parameters and the plurality of scanning parameters may be generated within the corresponding setting range. By combining the plurality of injection parameters and the plurality of scanning parameters, a series of candidate contrast scanning protocols may be generated. Subsequently, by comparing the enhancement effects of respective candidate contrast scanning protocols, the recommended contrast scanning protocol may be output, and the contrast injection parameters and the contrast scanning parameters in the recommended contrast scanning protocol may be displayed. For example, a schematic diagram of a contrast protocol recommendation interface is provided in FIG. 4, through which the content of the recommended contrast scanning protocol can be displayed.

In this embodiment, on one hand, the candidate contrast scanning protocols may be generated according to the setting range of contrast injection parameters and the setting range of contrast scanning parameters input by the user, and the user can be allowed to set recommendation ranges for respective parameters in a contrast scanning protocol in advance. In this way, the obtained candidate contrast scanning protocols and the recommended contrast scanning protocol may be ensured to satisfy practical requirements of the user while efficiently generating the plurality of candidate contrast scanning protocols. The possibility of abnormal parameters appearing in the contrast scanning protocol can be reduced, and the practicability and safety of the contrast scanning protocol can be improved. On the other hand, compared to the related art that only the recommended contrast agent injection parameters are considered, the candidate contrast scanning protocol including both the injection parameters and the scanning parameters can be generated in this embodiment. This enables a comprehensive consideration of the combined effect of the injection parameters and the scanning parameters, effectively improving and ensuring the consistency between the expected enhancement effect and the target enhancement effect. Furthermore, reasonable contrast imaging parameters and scanning parameters can be personalized and recommended. The enhancement effect can be ensured while the contrast agent dose can be minimized by the reasonable injection parameters matched with the target scanned object, and thus a renal metabolism burden of the patient can be reduced. The reasonable scanning parameters can yield higher-quality enhanced images while reducing a radiation dose.

In an embodiment, determining the recommended contrast scanning protocol from the plurality of candidate contrast scanning protocols according to the enhancement effect parameters of the plurality of candidate contrast scanning protocols may include the following steps: determining a preset target enhancement effect parameter, obtaining similarities between the enhancement effect parameters of the candidate contrast scanning protocols and the target enhancement effect parameter, and designating a candidate contrast scanning protocol whose similarity satisfies a preset similarity condition as the recommended contrast scanning protocol.

In specific implementation, the target enhancement effect parameter may be preset by the user, which may characterize an image enhancement effect expected by the user on the target medical image after the contrast scanning.

Subsequently, after the enhancement effect parameters of the plurality of candidate contrast scanning protocols are obtained, the enhancement effect parameter of each of candidate contrast scanning protocols may be compared to the target enhancement effect parameter to obtain the similarity between the enhancement effect parameter of the candidate contrast scanning protocol and the target enhancement effect parameter.

Then, a candidate contrast scanning protocol with a corresponding similarity satisfying the preset similarity condition among the plurality of candidate contrast scanning protocols may be used as the recommended contrast scanning protocol.

In some exemplary embodiments, the preset similarity condition may be a highest similarity. For example, a candidate contrast scanning protocol with an enhancement effect parameter most similar to the target enhancement effect parameter may be determined from the plurality of candidate contrast scanning protocols as the recommended contrast scanning protocol. For another example, the preset similarity condition may have a highest similarity while exceeding a similarity threshold. The similarity threshold may be determined based on an empirical value or based on a desired level of the enhancement effect representation required by the user. If the user needs to obtain an enhancement effect that matches the desired level, a higher similarity threshold may be set. Alternatively, if the enhancement effect required by the user has a relatively great fault-tolerant space, a lower similarity threshold may be set.

In some exemplary embodiments, if there are enhancement effect parameters across multiple dimensions, the key enhancement effect parameter selected from the target enhancement effect parameters of the multiple dimensions by the user may be determined. Then, among the plurality of candidate contrast scanning protocols, a candidate contrast scanning protocol having an enhancement effect parameter in a corresponding dimension closest to the key enhancement effect parameter may be determined as the recommended contrast scanning protocol. Alternatively, for each of candidate contrast scanning protocols, a first comprehensive enhancement effect parameter of the candidate contrast scanning protocol may be determined according to the multiple-dimension enhancement effect parameters of the candidate contrast scanning protocol. On the other hand, a second comprehensive enhancement effect parameter may also be determined according to the target enhancement effect parameters across dimensions. Then, a protocol having a first comprehensive enhancement effect parameter closest to the second comprehensive enhancement effect parameter among the plurality of candidate contrast scanning protocols may be used as the recommended contrast scanning protocol.

In this embodiment, by comparing the enhancement effects of respective candidate contrast scanning protocols and the target enhancement effect and selecting the candidate contrast scanning protocol whose similarity satisfies the preset similarity condition as the recommended contrast scanning protocol, personalized contrast protocol recommendations based on the individual condition of the scanned object can be achieved while also satisfying requirements of the enhancement effect for the user. This method balances both the adaptability and practicality of the obtained recommended contrast scanning protocol.

In an embodiment, determining the preset target enhancement effect parameter may include the following steps: determining the target enhancement effect parameters according to at least one of an imaging enhancement value, image quality evaluation information, or an image signal-to-noise ratio corresponding to a target medical image. The target medical image is a medical image that is expected to be obtained after performing contrast scanning based on a candidate contrast scanning protocol corresponding to the target medical image.

In practical applications, a to-be-obtained target enhancement effect parameter may be set by the user. For example, the to-be-obtained target enhancement effect parameter may be obtained by setting one or more pieces of the following information of the target medical image: the imaging enhancement value, the image quality evaluation information, and the image signal-to-noise ratio.

The image quality evaluation information may include information configured to evaluate a medical image from one or more dimensions. For example, the image quality evaluation information may include at least one image quality evaluation parameter, such as one or more of resolution, image contrast, and the like. The image quality evaluation information may also include an image quality evaluation score or an image quality evaluation level determined based on a single dimension or a plurality of comprehensive dimensions. Alternatively, the image quality evaluation information may further include other natural language statements configured to evaluate image quality.

In some exemplary embodiments, the user may input specific numerical values for the imaging enhancement value (also referred to as an image HU value), the image quality score, and the image signal-to-noise ratio. For example, an interface shown in FIG. 2 may be provided to the user, allowing the user to input an imaging enhancement value of A, an image quality score of B, and an image signal-to-noise ratio of C. It should be understood that the user may input all or part of the above information via the interface. Subsequently, the target enhancement effect parameter may be obtained by the device based on the information input by the user.

In this embodiment, by providing at least one of the imaging enhancement value, the image quality evaluation information, or the image signal-to-noise ratio of the target medical image, whether the enhancement effect of each of candidate contrast scanning protocols satisfies the requirements can be identified more accurately through quantifiable information. Thus, the impact of subjective human factors can be reduced, and the efficiency of obtaining the recommended contrast scanning protocol as well as the effectiveness of the recommended contrast scanning protocol can be improved.

In an exemplary embodiment, a training process of the contrast simulation model may be as follows: determining a sample contrast scanning protocol configured for a sample scanned object to acquire an expected image enhancement effect, and determining, according to an actual enhancement effect obtained when performing sample contrast scanning on the sample scanned object based on the sample contrast scanning protocol, first enhancement effect evaluation information that is taken as a label; inputting sample vital signs information of the sample scanned object, a sample enhancement effect parameter corresponding to the expected image enhancement effect, and the sample contrast scanning protocol into an original simulation model to obtain second enhancement effect evaluation information output by the original simulation model; and adjusting the original simulation model according to a difference between the second enhancement effect evaluation information and the first enhancement effect evaluation information until a training end condition is satisfied, to obtain the trained contrast simulation model.

In this embodiment, supervised training may be performed on the original simulation model by using sample vital signs information of the sample scanned object and the sample contrast scanning protocol, and the original simulation model can learn an application effect of each contrast scanning protocol on the scanned object and determine whether an expected image enhancement effect can be achieved, so that the model can quickly and accurately judge adaptation of multiple contrast scanning protocols to the scanned object, which facilitates improving subsequent flexible acquisition of a contrast scanning protocol that matches the scanned object.

In an exemplary embodiment, inputting the sample vital signs information of the sample scanned object, the sample enhancement effect parameter corresponding to the expected image enhancement effect, and the sample contrast scanning protocol into the original simulation model to obtain the second enhancement effect evaluation information output by the original simulation model, may further include: inputting the sample vital signs information of the sample scanned object, the sample enhancement effect parameter corresponding to the expected image enhancement effect, and the sample contrast scanning protocol into the original simulation model; and obtaining, by the original simulation model, sample vital signs information vectors corresponding to the sample scanned object, a sample enhancement effect vector corresponding to the sample enhancement effect parameter, and a contrast scanning protocol vector corresponding to the sample contrast scanning protocol, determining, by the original simulation model, a feature combination result according to the sample vital signs information vectors, and outputting, by the original simulation model, the second enhancement effect evaluation information according to the feature combination result, a sample enhancement effect feature corresponding to the sample enhancement effect vector, and a contrast scanning protocol feature corresponding to the contrast scanning protocol vector.

In an exemplary embodiment, the original simulation model may include an input layer, a processing layer, a hidden layer, and an output layer that are sequentially connected.

Obtaining, by the original simulation model, sample vital signs information vectors corresponding to the sample scanned object, the sample enhancement effect vector corresponding to the sample enhancement effect parameter, and the contrast scanning protocol vector corresponding to the sample contrast scanning protocol, may further include: obtaining, by the input layer, sample vital signs information vectors corresponding to the sample scanned object, the sample enhancement effect vector corresponding to the sample enhancement effect parameter, and the contrast scanning protocol vector corresponding to the sample contrast scanning protocol.

Determining, by the original simulation model, the feature combination result according to the sample vital signs information vectors, and outputting, by the original simulation model, the second enhancement effect evaluation information according to the feature combination result, the sample enhancement effect feature corresponding to the sample enhancement effect vector, and the contrast scanning protocol feature corresponding to the contrast scanning protocol vector, may further include: inputting the sample vital signs information vectors, the sample enhancement effect vector, and the contrast scanning protocol vector into the processing layer for feature extraction, and obtaining the feature combination result corresponding to the sample vital signs information vectors, the sample enhancement effect feature corresponding to the sample enhancement effect vector, and the contrast scanning protocol feature corresponding to the contrast scanning protocol vector; and inputting the feature combination result, the sample enhancement effect feature, and the contrast scanning protocol feature into the hidden layer for feature fusion, inputting a feature fusion result into the output layer, and obtaining the second enhancement effect evaluation information output by the output layer.

In an exemplary embodiment, the feature combination result may include features acquired in at least one of the following manners: determining consecutive features according to sample vital signs information vectors of first-type sample vital signs information in a plurality of pieces of sample vital signs information; encoding sample vital signs information vectors of second-type sample vital signs information in the plurality of pieces of sample vital signs information to obtain a discrete feature according to an encoding result; performing cross-combination on sample vital signs information vectors of third-type sample vital signs information in the plurality of pieces of sample vital signs information to obtain a cross-combination feature according to a cross-combination result; or performing feature extraction on sample vital signs information vectors of fourth-type sample vital signs information in the plurality of pieces of sample vital signs information to obtain a deep feature.

In an exemplary embodiment, the contrast simulation model may be obtained by the following step 201 to step 203.

Step 201 may include obtaining a sample scanned object physiological model of a sample scanned object, and obtaining actual enhancement effect parameters corresponding to a medical image obtained from the sample scanned object according to a sample contrast protocol.

The sample scanned object physiological model may be a physiological model for modeling and simulating physiological processes of the sample scanned object.

In specific implementation, the sample scanned object may be determined. The sample scanned object may refer to any scanned object undergone contrast and scanning according to actually performed sample contrast protocol. The sample contrast protocol may include injection parameters and scanning parameters.

After the sample scanned object is determined, a corresponding physiological model may be constructed for the sample scanned object to obtain the sample scanned object physiological model. The method of constructing this model may refer to the method of constructing the target scanned object physiological model, which will not be repeated here.

On the other hand, since the sample scanned object has been scanned and contrast imaged according to the corresponding sample contrast protocol, which means that a medical image corresponding to the scan with the sample contrast protocol is acquired, for example, a medical image obtained after scanning, contrast imaging, and reconstruction according to the sample contrast protocol. Thus, the enhancement effect parameters of the sample contrast protocol, i.e., the actual enhancement effect parameters, may also be determined according to the medical image.

Step 202 may include inputting the sample contrast protocol and the sample scanned object physiological model into an original simulation model, to obtain predicted enhancement effect parameters output by the original simulation model.

At this step, the sample contrast protocol and the sample scanned object physiological model may be input into the original simulation model. The original simulation model, based on the sample contrast protocol and the sample scanned object physiological model, may simulate the flow characteristics of the contrast agent in the sample scanned object and the enhancement condition of the target scanned region of the sample scanned object under the current scanning parameters. Then, the original simulation model may output the predicted enhancement effect parameters of the sample contrast protocol according to the result of the simulation.

Step 203 may include adjusting the original simulation model according to a difference between a predicted enhancement effect and an actual enhancement effect until a training end condition is satisfied, to obtain the trained contrast simulation model.

In this embodiment, the original simulation model may be trained through supervised learning. Specifically, a data set may be constructed, and each sample in the data set may include vital signs information of the sample scanned object, an actually performed sample contrast protocol, and a corresponding medical image. On one hand, the vital signs information of the sample scanned object may be used to construct the sample scanned object physiological model. On the other hand, a label value for the sample may be obtained based on the medical image. After the predicted enhancement effect output by the original simulation model is obtained, the difference between the predicted enhancement effect and the actual enhancement effect may be determined. Model parameters of the original simulation model may be then adjusted based on this difference. When the training end condition is not satisfied, the above steps may be repeatedly performed by using other samples in the data set, and the model parameters may be iteratively adjusted until the training end condition is satisfied. For example, when the difference between the predicted enhancement effect and the actual enhancement effect is less than a difference threshold, it may be determined that the training end condition is satisfied. The current simulation model may then be determined as the trained contrast simulation model.

In this embodiment, by inputting the sample contrast protocol and the sample scanned object physiological model into the original simulation model and adjusting the original simulation model according to the difference between the predicted enhancement effect parameters output by the original simulation model and the actual enhancement effect parameters, the original simulation model can learn how to accurately simulate the application condition of the contrast protocol on the scanned object through supervised learning. Thus, a data foundation can be provided for predicting reliable and accurate enhancement effects.

It should be noted that both the foregoing two training processes of the original simulation model may obtain the contrast simulation model. The original simulation model may be trained by either or both of the two training processes, and the obtained contrast simulation model may be applied in the present disclosure. In some embodiments, the original simulation model may also be trained to output the recommended contrast scanning protocol. Meanwhile the training process of the original simulation model may also be changed, which is not repeated herein.

To enable those skilled in the art to better understand the above steps, an example will be provided below to illustratively explain the embodiments of the present disclosure. However, it should be understood that the embodiments of the present disclosure are not limited thereto.

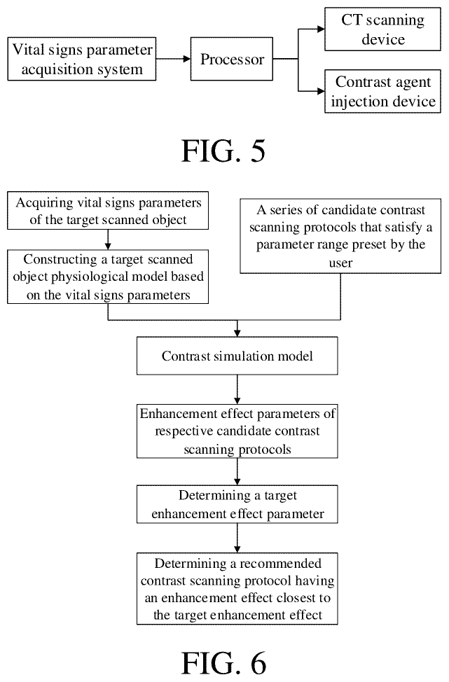

Referring to FIG. 5, a schematic structural diagram of an enhanced contrast scanning system is provided. The system includes a vital signs parameter acquisition system, a processor, a CT scanning device, and a contrast agent injection device. In this example, the vital signs parameter acquisition system may automatically measure and automatically read, or manually input vital signs parameters of the target scanned object, and provide the vital signs parameters to the processor. As illustrated in the flowchart of FIG. 6, the processor may construct a target scanned object physiological model based on the vital signs parameters, input the target scanned object physiological model and a series of candidate contrast scanning protocols that satisfy a parameter range preset by the user into the contrast simulation model to obtain enhancement effects of respective candidate contrast scanning protocols, and take a candidate contrast scanning protocol having an enhancement effect closest to a target enhancement effect as a recommended contrast scanning protocol. Subsequently, the processor may transmit injection parameters in the recommended contrast scanning protocol to the contrast agent injection device, and transmit scanning parameters in the recommended contrast scanning protocol to the CT scanning device. The contrast agent injection device may receive the injection parameters transmitted by the processor, and execute a contrast agent injection process based on the injection parameters. The CT scanning device may receive the scanning parameters and execute a CT scanning process based on the scanning parameters.

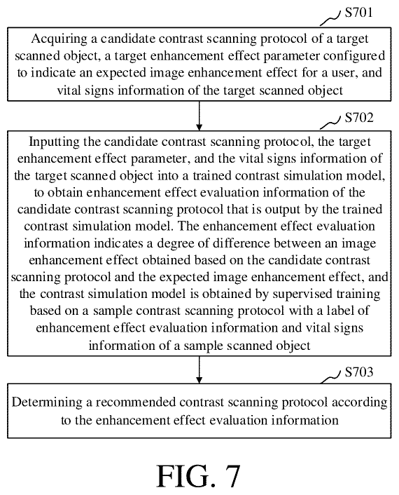

In an embodiment, referring to FIG. 7, a method of determining a contrast scanning protocol is provided. This embodiment is described by using an example in which the method is applied to a terminal device. In some examples, the terminal device may be a medical imaging device, such as a Computed Tomography (CT) device, a Magnetic Resonance Imaging (MRI) device, an Ultrasonic Contrast imaging device, or the like. It may be understood that the method may also be applied to a server, or may be applied to a system including a terminal and a server and implemented through interaction between the terminal and the server. In this embodiment, the method of determining the contrast scanning protocol includes the following step 701 and step 702.

Step 701 includes obtaining a candidate contrast scanning protocol of a target scanned object, a target enhancement effect parameter configured to indicate an expected image enhancement effect for a user, and vital signs information of the target scanned object.

The target scanned object may be any object to be injected with a contrast agent for scanning.

In specific implementation, one or more contrast scanning protocols may be set for the target scanned object, for example, multiple contrast scanning protocols may be set for comparison and screening by the user. For ease of distinguishing, multiple to-be-selected contrast scanning protocols provided may be referred to as candidate contrast scanning protocols. It may be understood that multiple different candidate imaging scanning protocols may be obtained by changing one or more contrast imaging parameters configured to control a contrast imaging manner. In some embodiments, the candidate contrast scanning protocol may be manually set, for example, the contrast imaging parameters may be manually set by the physicians according to an actual situation. Certainly, the candidate contrast scanning protocol may be automatically generated by a computer device. For example, a contrast scanning protocol generation model may be pre-trained. The contrast scanning protocol generation model may automatically generate and output a corresponding candidate contrast scanning protocol according to a contrast purpose and related parameters of the target scanned object.

In another aspect, the target enhancement effect parameter may be acquired. Specifically, the user may obtain different medical image enhancement effects by adjusting the contrast scanning protocols. In this embodiment, the user may preset the target enhancement effect parameter, and the expected image enhancement effect to be obtained for the user may be represented by the target enhancement effect parameter. The expected image enhancement effect may be an enhancement effect expected by the user, and may be used to indicate a degree of a difference between the lesion tissue and the normal tissue on an expected target medical image after the target scanned object is subjected to enhanced scanning by using the candidate contrast scanning protocol, or a degree of prominence of an imaging effect for the lesion tissue. A stronger expected image enhancement effect can lead to clearer boundaries between the lesion tissue and the normal tissue and allow the display of the density, the shape, the size, and the like of the lesion tissue more prominent.

In this embodiment, the target enhancement effect parameter may be specifically used to represent the expected image enhancement effect of the target medical image expected by the user. By setting one or more target enhancement effect parameters, the expected image enhancement effect that is to be acquired for the user may be represented from specific different aspects. In some examples, the target enhancement effect parameter may include an indicator parameter that can evaluate an expected image enhancement effect, and may include quantitative information, for example, a specific image parameter may be used to represent a degree of difference between the lesion tissue and the normal tissue, or may include qualitative information, for example, a level may be used to represent the degree of the prominence of the lesion tissue relative to the normal tissue.

Step 702 includes inputting the candidate contrast scanning protocol, the target enhancement effect parameter, and the vital signs information of the target scanned object into a trained contrast simulation model, to obtain enhancement effect evaluation information of the candidate contrast scanning protocol that is output by the trained contrast simulation model. The enhancement effect evaluation information indicates a degree of difference between an image enhancement effect obtained based on the candidate contrast scanning protocol and the expected image enhancement effect, and the contrast simulation model is obtained by supervised training based on a sample contrast scanning protocol with a label of enhancement effect evaluation information and vital signs information of a sample scanned object.

In specific implementation, vital signs information of the target scanned object may further be obtained, and the vital signs information may represent a physiological status of the scanned object. Then, the candidate contrast scanning protocol, the target enhancement effect, and the vital signs information of the target scanned object may be input into the trained contrast simulation model to obtain the enhancement effect evaluation information of the candidate contrast scanning protocol output by the trained contrast simulation model.

In addition, when there are a plurality of candidate contrast scanning protocols, the plurality of candidate contrast scanning protocols may be sequentially input into the trained contrast simulation model, or may be input into the trained contrast simulation model in batches.

Specifically, for a same candidate contrast scanning protocol, enhancement effects that can be acquired from different scanned objects may be different due to impact of various individual factors. In other words, when a same candidate contrast scanning protocol is applied on different scanned objects, and a degree of finally approaching the expected image enhancement effect may also be different.

In this embodiment, supervised training may be performed on the contrast simulation model by the sample contrast scanning protocol with the label of enhancement effect evaluation information and the vital signs information of the sample scanned object, so that the contrast simulation model may learn the enhancement effect when the contrast scanning protocol is applied to a specific scanned object, and output matching enhancement effect evaluation information. Furthermore, by inputting the candidate contrast scanning protocol, the target enhancement effect parameter, and the vital signs information of the target scanned object into the trained contrast simulation model, an analysis can be performed by the contrast simulation model. When the candidate contrast scanning protocol is applied to the target scanned object with specific vital signs information, the degree of difference or proximity between an image enhancement effect acquired based on the candidate contrast scanning protocol and the expected image enhancement effect indicated by the target enhancement effect parameter may be predicted, and corresponding enhancement effect evaluation information may be output for representation.

In some examples, the enhancement effect evaluation information may be understood as an evaluation result obtained by evaluating the image enhancement effect based on the candidate contrast scanning protocol, taking the expected image enhancement effect indicated by the target enhancement effect parameter as a reference. The enhancement effect evaluation information may indicate whether the image enhancement effect based on the candidate contrast scanning protocol can reach the expected image enhancement effect or indicate a degree of difference from the expected image enhancement effect for the user. In some alternative embodiments, the enhancement effect evaluation information may include an enhancement effect score. The enhancement effect score may represent a degree of the image enhancement effect obtained based on the candidate contrast scanning protocol reaching the expected image enhancement effect. For example, when the image enhancement effect predicted by the model can reach various target enhancement effect parameters set by the user, the enhancement effect score may be a full score. For another example, when the image enhancement effect predicted by the model partially reaches an enhancement degree indicated by the target enhancement effect parameters, such as the image enhancement effect reaches an enhancement degree indicated by a part of the target enhancement effect parameters, the enhancement effect score may not be a full score.

In this embodiment, a plurality of candidate contrast scanning protocols may be acquired, and enhancement effect evaluation information of each of the plurality of candidate contrast scanning protocols may be determined by the contrast simulation model. In some embodiments, multiple candidate contrast scanning protocols may be set for a same target enhancement effect parameter. In other words, for different multiple candidate contrast scanning protocols, the user may expect to acquire the expected image enhancement effect and set the same target enhancement effect parameter. The contrast simulation model may output respective enhancement effect evaluation information for the multiple candidate contrast scanning protocols that are corresponding to the same target enhancement effect parameter. In some other embodiments, multiple different groups of target enhancement effect parameters may be set, and at least one candidate contrast scanning protocol may be set for each group of target enhancement effect parameters. Then, enhancement effect evaluation information of multiple candidate contrast scanning protocols corresponding to the target enhancement effect parameters may be determined by the contrast simulation model.

In some exemplary embodiments, the target enhancement effect parameter may be determined by the following steps: determining the target enhancement effect parameter according to at least one of an imaging enhancement value, image quality evaluation information, or an image signal-to-noise ratio of the target medical image. The target medical image may be a medical image that is expected to be obtained after contrast imaging is performed according to a candidate contrast scanning protocol corresponding to the target medical image.

In actual application, the user may set a to-be-obtained target enhancement effect parameter. Specifically, for example, one or more pieces of the following information of the target medical image may be set to obtain the to-be-obtained target enhancement effect parameter: the imaging enhancement value (also referred to as an image HU value), the image quality evaluation information, and the image signal-to-noise ratio.

The image quality evaluation information may include information configured to evaluate a medical image from one or more dimensions. For example, the image quality evaluation information may include at least one image quality evaluation parameter, such as one or more of resolution, image contrast, and the like. The image quality evaluation information may also include an image quality evaluation score or an image quality evaluation level determined based on a single dimension or a plurality of comprehensive dimensions. Alternatively, the image quality evaluation information may further include other natural language statements configured to evaluate image quality.

In some exemplary embodiments, the user may input specific numerical values for the imaging enhancement value (also referred to as an image HU value), the image quality score, and the image signal-to-noise ratio. For example, an interface shown in FIG. 2 may be provided to the user, allowing the user to input an imaging enhancement value of A, an image quality score of B, and an image signal-to-noise ratio of C. It should be understood that the user may input all or part of the above information via the interface. Subsequently, the target enhancement effect parameter may be obtained by the device based on the information input by the user. By providing at least one of the imaging enhancement value, the image quality evaluation information, or the image signal-to-noise ratio of the target medical image, it may facilitate quantizing, by quantizable information, the expected image enhancement effect to be obtained by the user from various aspects. Correspondingly, in some embodiments, when determining the enhancement effect evaluation information, the contrast simulation model may also predict each expected enhancement effect parameter when the candidate imaging scanning protocol is applied to the target scanned object. For the target enhancement effect parameter and the expected enhancement effect parameter in a same dimension, a parameter similarity between the target enhancement effect parameter and the expected enhancement effect parameter may be determined. Subsequently, the enhancement effect evaluation information may be determined according to the parameter similarity in each dimension.

Step 703 includes determining a recommended contrast scanning protocol according to the enhancement effect evaluation information.

At this step, after obtaining the enhancement effect evaluation information that is output by the contrast simulation model for the candidate contrast scanning protocol, a proper contrast scanning protocol may be determined according to the enhancement effect evaluation information as the recommended contrast scanning protocol.

In some exemplary embodiments, when the user provides a single candidate contrast scanning protocol, after the contrast simulation model outputs the candidate contrast scanning protocol, it may be determined whether the enhancement effect evaluation information corresponding to the candidate contrast scanning protocol satisfies a preset evaluation requirement. If yes, the candidate contrast scanning protocol may be determined as the recommended contrast scanning protocol.

The preset evaluation requirement may include that the enhancement effect score corresponding to the enhancement effect evaluation information satisfies the preset threshold. When the preset evaluation requirement is not satisfied, the candidate contrast scanning protocol may be determined as a non-recommended contrast scanning protocol.

In other embodiments, when the user provides multiple candidate contrast scanning protocols, after the contrast simulation model outputs multiple pieces of enhancement effect evaluation information respectively corresponding to the multiple candidate contrast scanning protocols, a candidate contrast scanning protocol closest to the expected image enhancement effect may be screened out as the recommended contrast scanning protocol by comparing the multiple pieces of enhancement effect evaluation information. For example, a candidate contrast scanning protocol with a highest enhancement effect score may be determined as the recommended contrast scanning protocol. Furthermore, it may be determined whether the highest enhancement effect score satisfies a preset evaluation requirement, if yes, the candidate contrast scanning protocol may be determined as a recommended contrast scanning protocol; if no, all the multiple candidate contrast scanning protocols may be determined as non-recommended contrast scanning protocols.

In some exemplary embodiments, after determining the recommended contrast scanning protocol, the user may select the entire recommended contrast scanning protocol as the target contrast scanning protocol applied to the target scanned object, or may select a part of the recommended contrast scanning protocol as the target contrast scanning protocol applied to the target scanned object. For example, an injection parameter part of the recommended contrast scanning protocol may be selected, and a scanning parameter part may be manually adjusted by the user, so that the target contrast scanning protocol of the target scanned object may include the injection parameter part and the adjusted scanning parameter. The scanning parameter part may be manually adjusted by the user, so that the contrast scanning protocol may be more flexible in actual application.

In the foregoing method of determining the contrast scanning protocol, the candidate contrast scanning protocol of the target scanned object, the target enhancement effect parameter configured to indicate an expected image enhancement effect for the user, and the vital signs information of the target scanned object are acquired. Then, the candidate contrast scanning protocol, the target enhancement effect parameter, and the vital signs information of the target scanned object are input into the trained contrast simulation model to obtain the enhancement effect evaluation information of the candidate contrast scanning protocol output by the trained contrast simulation model. The enhancement effect evaluation information indicates the degree of difference between the image enhancement effect obtained based on the candidate contrast scanning protocol and the expected image enhancement effect, and the contrast simulation model is obtained by supervised training based on the sample contrast scanning protocol with the label of enhancement effect evaluation information and the vital signs information of the sample scanned object. Furthermore, the recommended contrast scanning protocol is determined according to the enhancement effect evaluation information. In this embodiment, supervised training is performed on the contrast simulation model by using the sample contrast scanning protocol with the label of enhancement effect evaluation information and the vital signs information of the sample scanned object, so that the contrast simulation model can learn the correlation between the enhancement effect of each contrast scanning protocol and the vital signs information of the sample scanned object. After the candidate contrast scanning protocol, the target enhancement effect parameter, and the vital signs information of the target scanned object are input into the contrast simulation model, the contrast simulation model can correctly predict the image enhancement effect of the candidate contrast scanning protocol when the candidate contrast scanning protocol is applied to the target scanned object, determine whether the enhancement effect to be obtained for the user can be satisfied, and output the enhancement effect evaluation information, so that the enhancement effect of each contrast scanning protocol for the target scanned object can be quickly determined, and it may facilitate accurately setting an appropriate contrast scanning protocol for the target scanned object, thereby improving a matching degree between the contrast scanning protocol and the target scanned object.

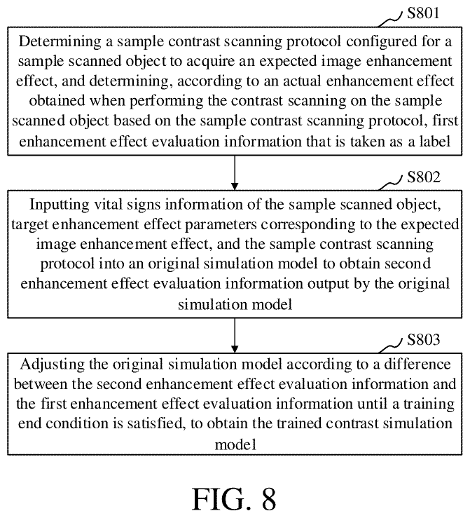

In some exemplary embodiments, referring to FIG. 8, a training process of the contrast simulation model may include the following step 801 to step 803.

Step 801 may include determining a sample contrast scanning protocol configured for a sample scanned object to acquire an expected image enhancement effect, and determining, according to an actual enhancement effect obtained when performing the contrast scanning on the sample scanned object based on the sample contrast scanning protocol, first enhancement effect evaluation information that is taken as a label.

In actual application, the sample scanned object may be determined. The sample scanned object may be any object that has been scanned according to a contrast scanning protocol to obtain an expected image enhancement effect. Since the sample scanned object has a corresponding medical image, i.e., a medical image obtained after scanning according to the contrast scanning protocol, this embodiment may further determine enhancement effect evaluation information according to an existing medical image of the sample scanned object. The existing medical image of the sample scanned object may refer to a medical image that has been generated after the sample scanned object is scanned before.

For ease of distinguishing, a contrast scanning protocol applied to the sample scanned object during contrast scanning may be referred to as the sample contrast scanning protocol. The sample contrast scanning protocol may include an injection parameter and a scanning parameter. The enhancement effect evaluation information determined according to the sample contrast scanning protocol may be referred to as first enhancement effect evaluation information, and the first enhancement effect evaluation information may be used as a label for supervised training of the model.

Step 802 may include inputting vital signs information of the sample scanned object, target enhancement effect parameters corresponding to the expected image enhancement effect, and the sample contrast scanning protocol into an original simulation model to obtain second enhancement effect evaluation information output by the original simulation model.

In actual application, the target enhancement effect parameter corresponding to the expected image enhancement effect may be determined. In this embodiment, the existing medical image of the sample scanned object may be obtained according to the sample contrast scanning protocol. Therefore, the target enhancement effect parameter may be determined according to the existing medical image. For example, the existing medical image of the sample scanned object may be analyzed to determine at least one of the image enhancement value, the image quality evaluation information, and the image signal-to-noise ratio corresponding to the existing medical image as the target enhancement effect parameter. Furthermore, the target enhancement effect parameter may be better than an enhancement effect parameter corresponding to the existing medical image.

In addition, vital signs information of the sample scanned object may be obtained, and the vital signs information may include vital signs information recorded when the contrast scanning is performed on the sample scanned object. Then, the vital signs information of the sample scanned object, the target enhancement effect parameter, and the corresponding sample contrast scanning protocol may be input into the original simulation model. Exemplarily, the original simulation model may include a model constructed based on a machine learning algorithm, such as a neural network model.

After obtaining the input information, the original simulation model may predict, according to the input information, an enhancement effect obtained by applying the sample contrast scanning protocol to the sample scanned object, determine a degree of difference or similarity between the enhancement effect and the expected image enhancement effect corresponding to the target enhancement effect parameter, and output corresponding enhancement effect evaluation information. For ease of distinguishing, the enhancement effect evaluation information may be referred to as second enhancement effect evaluation information. It should be noted that, when the enhancement effect is predicted, real scanning may not be performed by the sample contrast scanning protocol, but prediction may be performed based on the sample contrast scanning protocol.

Step 803 may include adjusting the original simulation model according to a difference between the second enhancement effect evaluation information and the first enhancement effect evaluation information until a training end condition is satisfied, to obtain the trained contrast simulation model.

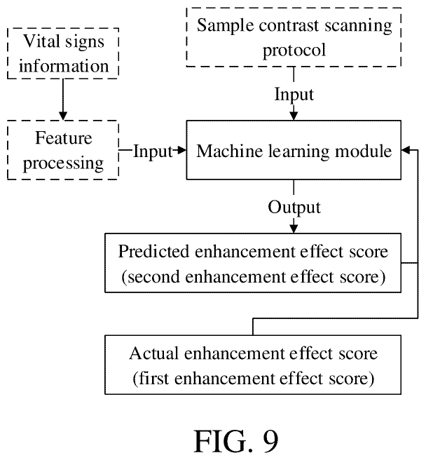

After the second enhancement effect evaluation information output by the original simulation model is obtained, the original simulation model may be optimized in a supervised learning manner. Specifically, referring to FIG. 9, the first enhancement effect evaluation information may be used as a label, and a difference between the second enhancement effect evaluation information and the first enhancement effect evaluation information may be determined. For example, when the enhancement effect evaluation information includes an enhancement effect score, a difference between a second enhancement effect score and a first enhancement effect score may be determined. Then, model parameters of the original simulation model may be adjusted according to the difference between the second enhancement effect evaluation information and the first enhancement effect evaluation information. After the adjustment of the model parameters is performed once, the foregoing steps may be repeatedly performed based on multiple training samples in the data set, and the model parameters may be iteratively adjusted until the training end condition is satisfied to obtain the trained contrast simulation model. For example, the training may be terminated when the difference between the second enhancement effect score output by the model and the first enhancement effect score is less than a threshold. Each of the multiple training samples in the data set may include vital signs information of the sample scanned object, a sample contrast scanning protocol, and first enhancement effect evaluation information.

In this embodiment, the supervised training may be performed on the original simulation model by using vital signs information of the sample scanned object and the sample contrast scanning protocol, so that the original simulation model can learn an application effect of each contrast scanning protocol on the scanned object and determine whether the expected image enhancement effect is achieved, and the model can quickly and accurately determine adaptation of multiple contrast scanning protocols to the scanned object, which facilitates improving subsequent flexible acquisition of the contrast scanning protocol that matches the scanned object.

In other exemplary embodiments, in addition to determining the enhancement effect evaluation information according to objective information (e.g., a degree of difference between a predicted image enhancement effect of the candidate contrast scanning protocol and the expected image enhancement effect), the original simulation model may further determine the enhancement effect evaluation information with reference to subjective information of an expert.

In some examples, the original simulation model may include an expert evaluation module and a data analysis module. The data analysis module may refer to a model obtained by training according to objective enhancement effect evaluation information. For example, the enhancement effect evaluation information used during training may be determined according to an actual enhancement effect obtained when the sample scanned object is scanned based on the sample contrast scanning protocol. For example, for the existing medical image obtained according to the sample contrast scanning protocol, parameter analysis may be performed to obtain the enhancement effect evaluation information. Therefore, the data analysis module may be understood that the enhancement effect evaluation information is predicted according to the objective information. For ease of distinguishing, the enhancement effect evaluation information output by the data analysis module may be referred to as the first enhancement effect evaluation information. The expert evaluation module may refer to a model obtained by training according to subjective enhancement effect evaluation information of the expert. For example, for the existing medical image obtained according to the sample contrast scanning protocol, the expert may give corresponding enhancement effect evaluation information according to expert experience, and then the enhancement effect evaluation information provided by the expert may be used as a label for training the model. For ease of distinguishing, the enhancement effect evaluation information output by the expert evaluation module may be referred to as the second enhancement effect evaluation information.

Furthermore, after the candidate contrast scanning protocol, the target enhancement effect parameter, and the vital signs information of the target scanned object are input into the trained contrast simulation model, the data analysis module may output the first enhancement effect evaluation information and the expert evaluation module may output the second enhancement effect evaluation information, and weighted summation processing may be performed according to the first enhancement effect evaluation information, the second enhancement effect evaluation information, and respective weights, to obtain the enhancement effect evaluation information of the candidate contrast scanning protocol, which is output as a processing result of the contrast simulation model.

The weights of the first enhancement effect evaluation information and the second enhancement effect evaluation information may be set by the user according to a requirement, or may be determined according to accuracy of the enhancement effect evaluation information output by the two modules. For example, a sample contrast scanning protocol that is configured to check the accuracy of the module may be input into the data analysis module and the expert evaluation module respectively to obtain the first enhancement effect evaluation information and the second enhancement effect evaluation information, and then a first similarity between the first enhancement effect evaluation information and the enhancement effect evaluation information used as the label may be acquired, and a second similarity between the second enhancement effect evaluation information and the enhancement effect evaluation information used as the label may be acquired. Respective weights of the first enhancement effect evaluation information and the second enhancement effect evaluation information may be determined according to a ratio of the first similarity to the second similarity.

In some examples, the expert evaluation module may further include multiple expert evaluation submodules. Different expert evaluation submodules may be trained according to enhancement effect evaluation information provided by experts in different professional fields, respectively. For example, experts in professional fields such as cardio-surgery, brain, and digestion may provide enhancement effect evaluation information for a medical image in a corresponding professional field (i.e., the existing medical image obtained according to the sample contrast scanning protocol), respectively. The expert evaluation module may learn common information and feature information in different professional fields, which facilitates improving accuracy of evaluation on contrast scanning protocols in various professional fields.

In an exemplary embodiment, at step 802, inputting the vital signs information of the sample scanned object, the target enhancement effect parameters corresponding to the expected image enhancement effect, and the sample contrast scanning protocol into the original simulation model to obtain the second enhancement effect evaluation information output by the original simulation model, may further include: inputting the vital signs information of the sample scanned object, the target enhancement effect parameter corresponding to the expected image enhancement effect, and the sample contrast scanning protocol into the original simulation model; and the original simulation model obtaining vital signs information vectors corresponding to the sample scanned object, a target enhancement effect vector corresponding to the target enhancement effect parameter, and a contrast scanning protocol vector corresponding to the sample contrast scanning protocol, determining a feature combination result according to the vital signs information vectors, and outputting the second enhancement effect evaluation information according to the feature combination result, a target enhancement effect feature corresponding to the target enhancement effect vector, and a contrast scanning protocol feature corresponding to the contrast scanning protocol vector.

In specific implementation, after the vital signs information of the sample scanned object, the target enhancement effect parameter corresponding to the expected image enhancement effect, and the sample contrast scanning protocol are input into the original simulation model, the model may first perform vector processing on the vital signs information, the target enhancement effect parameter, and the sample contrast scanning protocol to obtain the vital signs information vectors corresponding to the sample scanned object, the target enhancement effect vector corresponding to the target enhancement effect parameter, and the contrast scanning protocol vector corresponding to the sample contrast scanning protocol.

After the vital signs information vectors, the target enhancement effect vector, and the contrast scanning protocol vector are obtained, the vector content may be less representative. For example, taking the vital signs information vectors as an example, each of the vital signs information vectors may represent physiological information of the sample scanned object from different dimensions, but there may be a case of redundancy or contradiction between different vital signs information vectors. Meanwhile, some of original vital signs information vectors may be less representative. Similarly, the target enhancement effect vector and the contrast scanning protocol vector may also have redundant information or interference information.

In this regard, the original simulation model may perform feature extraction, acquire the feature combination result according to multiple vital signs information vectors, and acquire a target enhancement effect feature corresponding to the target enhancement effect vector and a contrast scanning protocol feature corresponding to the contrast scanning protocol vector. Furthermore, the second enhancement effect evaluation information may be output according to the feature combination result, the target enhancement effect feature, and the contrast scanning protocol feature.

In this embodiment, on one hand, by obtaining the vital signs information vectors corresponding to the sample scanned object, the target enhancement effect vector corresponding to the target enhancement effect parameter, and the contrast scanning protocol vector corresponding to the sample scanning protocol via the original simulation model, data of different modes (the vital signs information, the target enhancement effect parameter, and the sample contrast scanning protocol) can be converted into a uniform representation form, so that the original simulation model can simultaneously process multiple types of information in a same mathematical model, thereby improving reliability and accuracy of the second enhancement effect evaluation information. On the other hand, the feature combination result may be determined according to the vital signs information vectors, so that multiple pieces of vital signs information can be organically combined, and the feature combination that is more representative and has generalized capability can be extracted while redundancy information is removed, thereby improving prediction accuracy of the model.

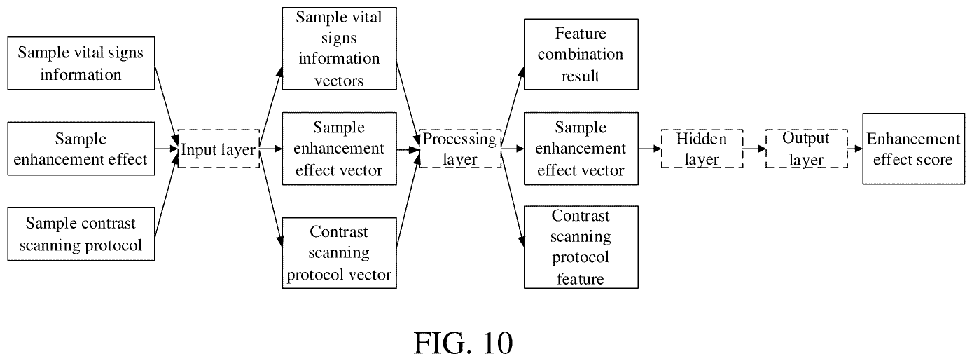

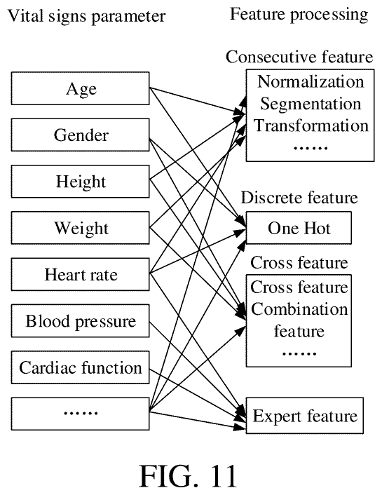

In an exemplary embodiment, referring to FIG. 10, a schematic structural diagram of the original simulation model is shown. The original simulation model may include an input layer, a processing layer, a hidden layer, and an output layer that are sequentially connected. The original simulation model obtaining vital signs information vectors corresponding to the sample scanned object, the target enhancement effect vector corresponding to the target enhancement effect parameter, and the contrast scanning protocol vector corresponding to the sample contrast scanning protocol, may further include: the input layer obtaining vital signs information vectors corresponding to the sample scanned object, the target enhancement effect vector corresponding to the target enhancement effect parameter, and the contrast scanning protocol vector corresponding to the sample contrast scanning protocol.

Correspondingly, determining the feature combination result according to the vital signs information vectors, and outputting the second enhancement effect evaluation information according to the feature combination result, the target enhancement effect feature corresponding to the target enhancement effect vector, and the contrast scanning protocol feature corresponding to the contrast scanning protocol vector, may further include: inputting the vital signs information vectors, the target enhancement effect vector, and the contrast scanning protocol vector into the processing layer for feature extraction, and obtaining the feature combination result corresponding to the vital signs information vectors, the target enhancement effect feature corresponding to the target enhancement effect vector, and the contrast scanning protocol feature corresponding to the contrast scanning protocol vector; and inputting the feature combination result, the target enhancement effect feature, and the contrast scanning protocol feature into the hidden layer for feature fusion, inputting a feature fusion result into the output layer, and obtaining the second enhancement effect evaluation information output by the output layer.

In actual application, after the vital signs information of the target scanned object, the target enhancement effect, and the multiple candidate contrast scanning protocols are obtained, referring to FIG. 10, for each of the sample contrast scanning protocols, the sample contrast scanning protocol, the vital signs information of the sample scanned object, and the target enhancement effect parameter that is obtained according to the sample contrast scanning protocol may be input into the input layer, to obtain the vital signs information vectors corresponding to the vital signs information, the target enhancement effect vector corresponding to the target enhancement effect parameter, and the contrast scanning protocol vector corresponding to the sample contrast scanning protocol which are output by the input layer.

Then, the vital signs information vectors, the target enhancement effect vector, and the contrast scanning protocol vector may be input into the processing layer to perform feature extraction, to obtain the feature combination result corresponding to the vital signs information vectors, the target enhancement effect feature corresponding to the target enhancement effect vector, and the contrast scanning protocol feature corresponding to the contrast scanning protocol vector. Then, the feature combination result, the target enhancement effect feature, and the contrast scanning protocol feature may be input into the hidden layer to perform feature fusion, and then the feature fusion result output by the hidden layer may be input into the output layer, to obtain the second enhancement effect evaluation information that is output by the output layer for the sample contrast scanning protocol.

In this embodiment, on one hand, the original simulation model can acquire the vital signs information vectors, the target enhancement effect vector, and the contrast scanning protocol vector by the input layer, and convert multi-modal input into an input in a uniform form. On another hand, the processing layer may perform feature extraction on the vital signs information vectors, the target enhancement effect vector, and the contrast scanning protocol vector, so that key information can be retained while a data computation amount is reduced, and a model expression capability is enhanced. On the other hand, the hidden layer may perform feature fusion on multiple extracted features and input the feature fusion result into the output layer to form a higher-level feature fusion result, which facilitates the model understanding an inherent rule of data and improving accuracy of the second enhancement effect evaluation information.

In some exemplary embodiments, the feature combination result may include features acquired in at least one of the following manners: determining consecutive features according to vital signs information vectors of first-type vital signs information in a plurality of pieces of vital signs information; encoding vital signs information vectors of second-type vital signs information in the plurality of pieces of vital signs information to obtain a discrete feature according to an encoding result; performing cross-combination on vital signs information vectors of third-type vital signs information in the plurality of pieces of vital signs information to obtain a cross-combination feature according to a cross-combination result; or performing feature extraction on vital signs information vectors of fourth-type vital signs information in the plurality of pieces of vital signs information to obtain a deep feature.

In specific implementation, the multiple pieces of vital signs information may include different types of data, for example, one or more of a continuous type, a discrete type, an enumeration type, or a text type. In this embodiment, to fully use the multiple pieces of vital signs information, multiple feature processing may be performed on the multiple pieces of vital signs information.

Specifically, first-type vital signs information may be determined from multiple pieces of vital signs information. The first-type vital signs information may refer to vital signs information that is of a value in consecutive values. Then, the consecutive feature may be determined according to the vital signs information vector of the first-type vital signs information. For example, the vital signs information vector of the first-type vital signs information may be input into the processing layer, and the consecutive feature may be obtained according to the output of the processing layer. In some examples, referring to FIG. 11, the first-type vital signs information may include one or more of an age, a height, a weight, a heart rate, and an ejection fraction of the target scanned object. During processing, the model may input the age, the height, the weight, the heart rate, and the ejection fraction of the target scanned object into the input layer, to obtain an age vector, a height vector, a weight vector, a heart rate vector, and an ejection fraction vector that are output by the input layer, and input the age vector, the height vector, the weight vector, the heart rate vector, and the ejection fraction vector into the processing layer, so that the processing layer may perform at least one operation of normalization, segmentation, or standardization on each input vector to obtain the consecutive feature.

In another aspect, second-type vital signs information may be determined from the multiple pieces of vital signs information. The second-type vital signs information may refer to vital signs information of a discrete value, such as a gender or medical record information. A discrete feature may be obtained by encoding a vital signs information vector corresponding to the second-type vital signs information. Taking the gender and the medical record information as an example, the gender and the medical record information of the target scanned object may be input to the input layer, to obtain a gender vector and a medical record information vector that are output by the input layer, and then the gender vector and the medical record information vector may be input to the processing layer, and the processing layer may perform One-Hot encoding on the input gender vector and the medical record information vector to obtain the discrete feature.

In addition, third-type vital signs information may be determined from the multiple pieces of vital signs information. The third-type vital signs information may refer to multiple pieces of vital signs information on which cross-combination processing is to be performed. The cross-combination processing may mean that a preset form of combination or cross-combination is performed on the original information in the processing layer to generate a new feature. The cross-combination processing may construct a cross feature based on a rule determined by artificial prior knowledge, or automatically mining association and interaction between the features by using an algorithm. After the third-type vital signs information is determined, a cross combination may be performed on vital signs information vectors of the third-type vital signs information to obtain the cross-combination result, and then the cross-combination result may be used as the cross combination feature. For example, for the gender, the height, and the weight of the target scanned object, the gender, the height, and the weight of the scanned object may be input into the input layer, to obtain the gender vector, the height vector, and the weight vector that are output by the input layer. The gender vector, the height vector, and the weight vector may be input into the processing layer, and the processing layer may perform cross-combination processing to obtain the cross-combination feature.

The fourth type of vital signs information may further be determined from the multiple pieces of vital signs information. The fourth type of vital signs information may refer to vital signs information in which an understanding result or an analysis result depends on specified professional knowledge, for example, cardiac function information, a positioning image, a flat scanning image, and the like. After the fourth-type vital signs information is determined, feature extraction may be performed on the vital signs information vector of the fourth-type vital signs information to obtain the deep feature. For example, a cardiac function, a positioning image, and a flat scanning image of the target scanned object may be obtained and input to the input layer, to obtain a cardiac function vector, a positioning image vector, and a flat scanning image vector that are output by the input layer. The cardiac function vector, the positioning image vector, and the flat scanning image vector may be input into the processing layer, the processing layer may perform feature extraction to obtain the deep feature, and the deep feature may also be referred to as an expert feature.

In this embodiment, different feature processing may be performed on the multiple pieces of vital signs information in multiple manners, so that key content in the vital signs information of the target scanned object can be fully excavated, and a physiological status of the target scanned object may be more accurately and comprehensively described. This may facilitate improving accuracy of the enhanced effect evaluation information output by the contrast simulation model, and improving a matching degree between the recommended contrast scanning protocol and a status of the scanned object.

It may be understood that, in actual application, a process in which the model outputs the first enhancement effect evaluation information may be the same as that in which the model outputs the second enhancement effect evaluation information, details may not be described in the present disclosure, and a manner of obtaining the first enhancement effect evaluation information may refer to content in the foregoing one or more embodiments.

In an exemplary embodiment, the method may further include: obtaining one or more optimized contrast scanning protocols according to protocol adjustment information entered by a user for the recommended contrast scanning protocol, and adjusting the trained contrast simulation model for updating according to differences between the one or more optimized contrast scanning protocols and the recommended contrast scanning protocol, to obtain an updated trained contrast simulation model that matches a protocol configuration habit of the user.

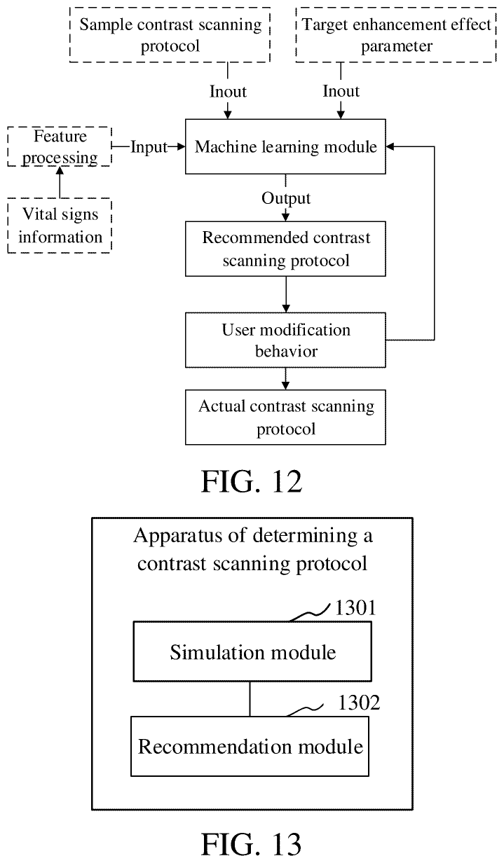

In specific implementation, when the user uses the contrast simulation model, the user may adjust the finally determined recommended contrast scanning protocol. In this case, the device may obtain the protocol adjustment information entered by the user for the recommended contrast scanning protocol, and determine, according to the protocol adjustment information, the optimized contrast scanning protocols adjusted by the user. For example, referring to FIG. 12, multiple candidate contrast scanning protocols may be obtained, and then the vital signs information of the target scanned object may be input into the model. After the enhancement effect score that is predicted and output by the model for the candidate contrast scanning protocol is obtained, the recommended contrast scanning protocol may be determined and provided to the user according to the enhancement effect score of each of the multiple candidate contrast scanning protocols. The user can adjust the recommended contrast scanning protocol to obtain the optimized contrast scanning protocols (also referred to as actual contrast scanning protocols).

Then, the contrast simulation model may be adjusted for updating according to differences between the optimized contrast scanning protocols and the recommended contrast scanning protocol, to obtain the contrast simulation model that matches the protocol configuration habit of the user. Specifically, in some cases, the finally obtained recommended contrast scanning protocol may have better enhancement effect evaluation information than other candidate contrast scanning protocols, but content of the recommended contrast scanning protocol may not meet expectation of the user. In this regard, in this embodiment, the difference between the optimal contrast scanning protocol and the recommended contrast scanning protocol may be determined, and the contrast simulation model may be adjusted, so that the adjusted model can provide higher enhancement effect evaluation information to a candidate contrast scanning protocol that is more compliant with the protocol configuration habit of the user, which facilitates subsequently screening out a contrast scanning protocol that is more compliant with the protocol configuration habit of the user from the multiple candidate contrast scanning protocols.

In an exemplary embodiment, each of the optimized contrast scanning protocols may be corresponding to a contrast purpose, which indicates a medical task applied to a medical image obtained after performing the contrast scanning based on the optimized contrast scanning protocol. For example, some optimized contrast scanning protocols may be set for angiography, others may be set for alimentary tract imaging, a medical image device used in some optimized contrast scanning protocols may be a CT device, and a medical image device used in some other optimized contrast scanning protocols may be a magnetic resonance device.

Correspondingly, adjusting the contrast simulation model for updating according to differences between the optimized contrast scanning protocols and the recommended contrast scanning protocol, to obtain the contrast simulation model that matches the protocol configuration habit of the user, may further include: determining a plurality of optimized contrast scanning protocols corresponding to the same contrast purpose; and for each contrast purpose, adjusting the contrast simulation model according to differences between the plurality of optimized contrast scanning protocols corresponding to the contrast purpose and a corresponding recommended contrast scanning protocol, to obtain the contrast simulation model that matches the protocol configuration habit of the user and the contrast purpose.

In specific implementation, for different contrast purposes, a manner of the user to adjust the contrast scanning protocol may be different. When a model is optimized for all contrast purposes in a same manner, matching between a subsequently obtained recommended contrast scanning protocol and a usage scenario may be affected.

In this embodiment, for each contrast purpose, optimized contrast scanning protocols corresponding to the contrast purpose may be determined. Then, for each of optimized contrast scanning protocols corresponding to the contrast purpose, a difference between the optimized contrast scanning protocol and a corresponding recommended contrast scanning protocol before adjustment by the user may be determined, and the contrast simulation model may be adjusted according to the difference. The model parameter may be iteratively adjusted according to the optimized contrast scanning protocols under the contrast purpose for multiple times until a training end condition is satisfied, to obtain a contrast simulation model that matches the contrast configuration habit of the user and the contrast purpose.

In this embodiment, the contrast simulation model may be adjusted according to differences between optimized contrast scanning protocols and the corresponding recommended contrast scanning protocol for each contrast purpose, which facilitates differentially determining an operation habit of the user in different contrast scenarios and providing a matched contrast simulation model.

In an exemplary embodiment, the method may further include the following steps: obtaining a blood flow feature that reflects a blood flow status of the target scanned object, and obtaining the vital signs information of the target scanned object according to the blood flow feature, a physical feature of the target scanned object, and historical abnormality information of the target scanned object.

In the related art, related attribute information of the scanned object may also be obtained, and a matching contrast protocol may be output by a regressive apparatus. In these manners, mainly reference patient attributes only include a weight, a body surface area, an age, and a height. However, the inventor finds, through practice, that there is still a relatively large space for improving matching between the contrast protocol acquired in these manners and the scanned object. After creative efforts, it is found that, when describing a physiological state of the scanned object, there is a lack of a vital signs parameter that reflects a condition in which the contrast agent flows in the body, thereby affecting adaptation between a predicted contrast protocol and the scanned object.

In this embodiment, the blood flow feature that reflects the blood flow status of the target scanned object may be obtained. In some examples, the blood flow feature may include at least one of the following information: a heart rate, a blood pressure, a cardiac function, or an ejection fraction. In addition, the physical feature that represents the body state of the target scanned object and the historical abnormality information that represents the past abnormality of the target scanned object may further be obtained. In some examples, the physical feature may include one or more of the following information: an age, a gender, a height, a weight, a positioning image, and a flat scanning image. The history abnormality information may include the medical record information.

Furthermore, the blood flow feature, the physical feature, and the historical abnormality information of the target scanned object may be used as the vital signs information of the target scanned object.

In this embodiment, the vital signs information of the target scanned object may be obtained according to the blood flow feature, the physical feature, and the historical abnormality information of the target scanned object. When data availability is considered, multiple pieces of vital signs information that have a key impact on the enhancement effect of the contrast protocol can be accurately and effectively obtained, which facilitates the model better predicting the enhancement effect of the candidate contrast scanning protocol.

In an exemplary embodiment, obtaining candidate contrast scanning protocols of the target scanned object may further include: determining injection parameters and scanning parameters, and obtaining the candidate contrast scanning protocols according to the injection parameters and the scanning parameters.

The injection parameters may include one or more parameters related to a drug injection in the contrast process, and a manner for the drug injection in the contrast process may be controlled by the injection parameters. In some exemplary embodiments, the injection parameters may include one or more of a contrast agent identifier, a contrast agent type, a contrast agent injection rate, a contrast agent injection duration, a contrast agent injection volume, a follower agent injection rate, a follower agent injection duration, and a follower agent injection volume.

The scanning parameters may include one or more parameters configured to control the scanning manner of the medical image device in the contrast process, and the scanning mode for the target scanned object in the contrast process may be controlled by the scanning parameters. In some exemplary embodiments, the scanning parameters may include one or more of the following parameters related to the enhancement effect: a tube voltage and a tube current. In addition, the scanning parameters may also include parameters such as a monitoring threshold and a scanning delay value. In some exemplary embodiments, several related scanning parameters may be provided to the user according to a scanning device selected by the user, then the setting range of contrast scanning parameters input by the user for each scanning parameter may be acquired, and one or more scanning parameters may be automatically obtained within this range. For example, when the scanning is triggered by means of Bolus-Tracking after injection of the contrast agent, recommended scanning parameter may include some or all of parameters including the monitoring threshold, a post-injection delay time, a post-threshold delay time, a Tracker interval time, and a scanning voltage.

In specific implementation, the injection parameters and the scanning parameters of the target scanned object may be obtained, and the candidate contrast scanning protocol to be screened may be obtained according to the injection parameters and the scanning parameters. The obtained candidate contrast scanning protocol may include the injection parameters and the scanning parameters. It may be understood that multiple different candidate imaging scanning protocols may be obtained by changing one or more of the injection parameters or the scanning parameters.

In some embodiments, the candidate contrast scanning protocol may be manually set. For example, the physician may manually set, according to an actual situation, the candidate contrast scanning protocol including the injection parameters and the scanning parameters. In other embodiments, the injection parameters and the scanning parameters may also be automatically generated by a computer device, to obtain the candidate contrast scanning protocol. For example, a contrast protocol generation model may be pre-trained, and the contrast protocol generation model may automatically generate and output, according to the contrast purpose and related parameters of the target scanned object, the candidate contrast scanning protocol including the injection parameters and the scanning parameters.

In some embodiments, a regressive apparatus may be established by a machine learning method, and the contrast scanning protocol of the target scanned object may be generated by the regressive apparatus. However, the contrast scanning protocol may mainly include parameters related to a contrast injection manner, and not provide a targeted and differentiated scanning manner for the target scanned object during the contrast scanning process. The inventor finds, in practice, that scanning all scanned objects in a same scanning manner may have different negative effects on the scanned object.

In this embodiment, the candidate contrast scanning protocol that includes the injection parameters and the scanning parameters may be acquired, and corresponding enhancement effect evaluation information may be output for each candidate contrast scanning protocol with subsequent reference to the contrast simulation model, and reasonable injection parameters and scanning parameters can be personally recommended according to the target scanned object. Reasonable injection parameters that match the target scanned object can ensure the enhancement effect while minimizing the dose of the contrast agent, thereby reducing the renal metabolism burden of the patient, and reasonable scanning parameters can help to obtain an enhanced image with higher quality while reducing the radiation dose. In addition, by generating the candidate contrast scanning protocol that includes the injection parameters and the scanning parameters, a combination effect of the injection parameters and the scanning parameters can be comprehensively considered, thereby effectively improving and ensuring the consistency between the expected enhancement effect and the actual enhancement effect.

It should be understood that, although steps in the flowchart related to the foregoing embodiments are sequentially displayed according to an instruction of an arrow, these steps are not necessarily sequentially performed according to an instruction of an arrow. Unless expressly stated in this specification, these steps are not performed in a strict order, and these steps may be performed in another order. In addition, at least a part of steps in the flowchart involved in the foregoing embodiments may include multiple steps or multiple phases. These steps or phases are not necessarily performed at a same moment, but may be performed at different moments. An execution sequence of these steps or phases is not necessarily performed sequentially, but may be performed alternately or alternately with at least a part of steps or phases in another step or another step.

Based on a same invention concept, an apparatus of determining a contrast scanning protocol configured to implement the foregoing method of determining the contrast scanning protocol is further provided in an embodiment of the present disclosure. An implementation solution provided by the apparatus is similar to the implementation solution described in the foregoing method. Therefore, a specific limitation in the apparatus embodiment of determining one or more contrast scanning protocols provided in the following may refer to the foregoing limitation on the method of determining the contrast scanning protocol. Details are not described herein again.

In an exemplary embodiment, referring to FIG. 13, an apparatus of determining a contrast scanning protocol is provided, including a simulation module 1301 and a recommendation module 1302.

The simulation module 1301 is configured for obtaining a target scanned object physiological model of a target scanned object, inputting the target scanned object physiological model and a plurality of candidate contrast scanning protocols into a trained contrast simulation model to obtain enhancement effect parameters of the plurality of candidate contrast scanning protocols.

The contrast simulation model is configured to simulate a flow characteristic of a contrast agent in the target scanned object and an enhancement condition of a target scanned region of the target scanned object under current scanning parameters, and output the enhancement effect parameters according to a result of the simulation.

The recommendation module 1302 is configured for determining a recommended contrast scanning protocol from the plurality of candidate contrast scanning protocols according to the enhancement effect parameters of the plurality of candidate contrast scanning protocols.

In an embodiment, the simulation module 1301 is configured for determining target scanned region information and target injection region information of the target scanned object, obtaining a general physiological model from a preset model library according to the target scanned region information and the target injection region information, and adjusting the general physiological model to obtain the target scanned object physiological model corresponding to the target scanned object. The general physiological model characterizes physiological structural information between the target scanned region and the target injection region.

In an embodiment, the simulation module 1301 is configured for performing vital signs information extraction on the target scanned object, and adjusting the general physiological model according to the extracted vital signs information to obtain the target scanned object physiological model corresponding to the target scanned object. The vital signs information includes either or both of physiological parameters affecting changes in fluid flow characteristics in the target scanned region and medical information of the target scanned object.

In an embodiment, the simulation module 1301 is configured for determining a setting range of contrast injection parameters and a setting range of contrast scanning parameters, determining a plurality of injection parameters in the setting range of contrast injection parameters and a plurality of scanning parameters in the setting range of contrast scanning parameters, and obtaining the plurality of candidate contrast scanning protocols according to a combination result of the plurality of injection parameters and the plurality of scanning parameters.

In an embodiment, the recommendation module 1302 is configured for determining a preset target enhancement effect parameter, obtaining similarities between the enhancement effect parameters of the candidate contrast scanning protocols and the target enhancement effect parameter, and designating a candidate contrast scanning protocol whose similarity satisfies a preset similarity condition as the recommended contrast scanning protocol.

In an embodiment, the recommendation module 1302 is configured for determining the target enhancement effect parameter according to at least one of an imaging enhancement value, image quality evaluation information, or an image signal-to-noise ratio corresponding to a target medical image. The target medical image is a medical image that is expected to be obtained after performing contrast scanning based on a candidate contrast scanning protocol corresponding to the target medical image.

In an embodiment, the apparatus may further include a model training module, which is configured for obtaining a sample scanned object physiological model of a sample scanned object, and obtaining actual enhancement effect parameters corresponding to a medical image obtained from the sample scanned object according to a sample contrast protocol; inputting the sample contrast protocol and the sample scanned object physiological model into an original simulation model, to obtain predicted enhancement effect parameters output by the original simulation model; and adjusting the original simulation model according to a difference between a predicted enhancement effect and an actual enhancement effect until a training end condition is satisfied, to obtain the trained contrast simulation model.

Based on a same invention concept, an apparatus of determining a contrast scanning protocol configured to implement the foregoing method of determining the contrast scanning protocol is further provided in an embodiment of the present disclosure. An implementation solution provided by the apparatus is similar to the implementation solution described in the foregoing method. Therefore, a specific limitation in the apparatus embodiment of determining one or more contrast scanning protocols provided in the following may refer to the foregoing limitation on the method of determining the contrast scanning protocol. Details are not described herein again.

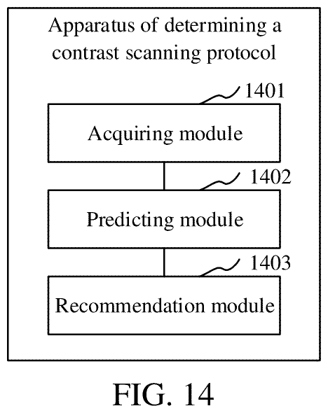

In an exemplary embodiment, referring to FIG. 14, an apparatus of determining a contrast scanning protocol is provided, including an obtaining module 1401, a predicting module 1402, and a recommendation module 1403.

The obtaining module 1401 is configured for obtaining a candidate contrast scanning protocol of a target scanned object, a target enhancement effect parameter configured to indicate an expected image enhancement effect for a user, and vital signs information of the target scanned object.

The predicting module 1402 is configured for inputting the candidate contrast scanning protocol, the target enhancement effect parameter, and the vital signs information of the target scanned object into a trained contrast simulation model, to obtain enhancement effect evaluation information of the candidate contrast scanning protocol that is output by the trained contrast simulation model. The enhancement effect evaluation information indicates a degree of difference between an image enhancement effect obtained based on the candidate contrast scanning protocol and the expected image enhancement effect, and the contrast simulation model is obtained by supervised training based on a sample contrast scanning protocol with a label of enhancement effect evaluation information and vital signs information of a sample scanned object.

The recommendation module 1403 is configured for determining a recommended contrast scanning protocol according to the enhancement effect evaluation information.

In an embodiment, the apparatus may further include a model training module, which is configured for determining a sample contrast scanning protocol configured for a sample scanned object to acquire an expected image enhancement effect, and determining, according to an actual enhancement effect obtained when performing the contrast scanning on the sample scanned object based on the sample contrast scanning protocol, first enhancement effect evaluation information that is taken as a label; inputting vital signs information of the sample scanned object, target enhancement effect parameters corresponding to the expected image enhancement effect, and the sample contrast scanning protocol into an original simulation model to obtain second enhancement effect evaluation information output by the original simulation model; and adjusting the original simulation model according to a difference between the second enhancement effect evaluation information and the first enhancement effect evaluation information until a training end condition is satisfied, to obtain a trained contrast simulation model.

In an embodiment, the model training module is configured for inputting the vital signs information of the sample scanned object, the target enhancement effect parameter corresponding to the expected image enhancement effect, and the sample contrast scanning protocol into the original simulation model; and the original simulation model obtaining vital signs information vectors corresponding to the sample scanned object, a target enhancement effect vector corresponding to the target enhancement effect parameter, and a contrast scanning protocol vector corresponding to the sample contrast scanning protocol, determining a feature combination result according to the vital signs information vectors, and outputting the second enhancement effect evaluation information according to the feature combination result, a target enhancement effect feature corresponding to the target enhancement effect vector, and a contrast scanning protocol feature corresponding to the contrast scanning protocol vector.

In an embodiment, the original simulation model includes an input layer, a processing layer, a hidden layer, and an output layer that are sequentially connected.

The model training module is configured for obtaining vital signs information vectors corresponding to the sample scanned object, the target enhancement effect vector corresponding to the target enhancement effect parameter, and the contrast scanning protocol vector corresponding to the sample contrast scanning protocol by the input layer; inputting the vital signs information vectors, the target enhancement effect vector, and the contrast scanning protocol vector into the processing layer for feature extraction, and obtaining the feature combination result corresponding to the vital signs information vectors, the target enhancement effect feature corresponding to the target enhancement effect vector, and the contrast scanning protocol feature corresponding to the contrast scanning protocol vector; and inputting the feature combination result, the target enhancement effect feature, and the contrast scanning protocol feature into the hidden layer for feature fusion, inputting a feature fusion result into the output layer, and obtaining the second enhancement effect evaluation information output by the output layer.

In an embodiment, the feature combination result includes features acquired in at least one of the following manners: determining consecutive features according to vital signs information vectors of first-type vital signs information in a plurality of pieces of vital signs information; encoding vital signs information vectors of second-type vital signs information in the plurality of pieces of vital signs information to obtain a discrete feature according to an encoding result; performing cross-combination on vital signs information vectors of third-type vital signs information in the plurality of pieces of vital signs information to obtain a cross-combination feature according to a cross-combination result; or performing feature extraction on vital signs information vectors of fourth-type vital signs information in the plurality of pieces of vital signs information to obtain a deep feature.

In an embodiment, the apparatus may further include a model optimizing module, which is configured for obtaining one or more optimized contrast scanning protocols according to protocol adjustment information entered by a user for the recommended contrast scanning protocol, and adjusting the trained contrast simulation model for updating according to differences between the one or more optimized contrast scanning protocols and the recommended contrast scanning protocol, to obtain an updated trained contrast simulation model that matches a protocol configuration habit of the user.

In an embodiment, the obtaining module 1401 is configured for determining injection parameters and scanning parameters, and obtaining the candidate contrast scanning protocol according to the injection parameters and the scanning parameters.

The modules in the above apparatus of determining the contrast scanning protocol may be implemented entirely or partially through software, hardware, or a combination thereof. The above modules may be embedded in or independent of the processor in a computer device in the form of hardware, or may be stored in a memory in the computer device in the form of software, so that the processor can invoke and perform the operations corresponding to the above modules.

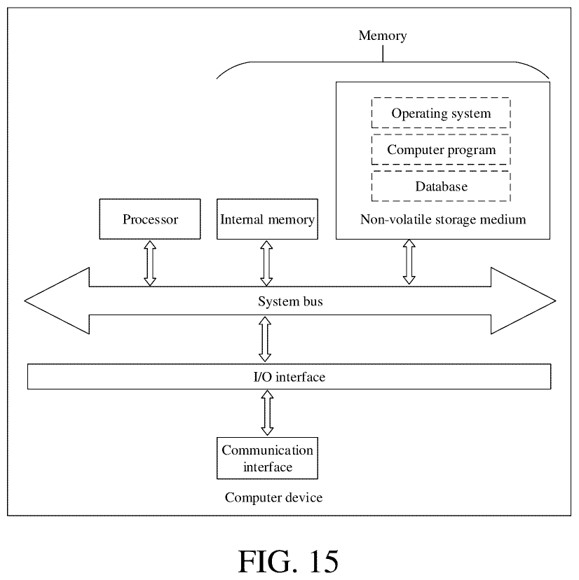

In an exemplary embodiment, a computer device is provided. The computer device may be a server, and an internal structure diagram of the computer device may be shown in FIG. 15. The computer device includes a processor, a memory, an input/output (I/O) interface, and a communications interface. The processor, the memory, and the input/output interface are connected via a system bus, and the communications interface is connected to the system bus via the input/output interface. The processor of the computer device is configured to provide computing and control capabilities. The memory of the computer device includes a non-volatile storage medium and an internal memory. The non-volatile storage medium stores an operating system, a computer program, and a database. The internal memory provides an environment for the operation of the operating system and the computer program in the non-volatile storage medium. The database of the computer device is configured to store vital signs data of the scanned object, contrast simulation models, target scanned object physiological models, and the like. The input/output interface of the computer device is configured to exchange information between the processor and an external device. The communication interface of the computer device is configured to communicate with an external terminal through a network connection. The computer program is executed by the processor to implement a method for obtaining a contrast scanning protocol.

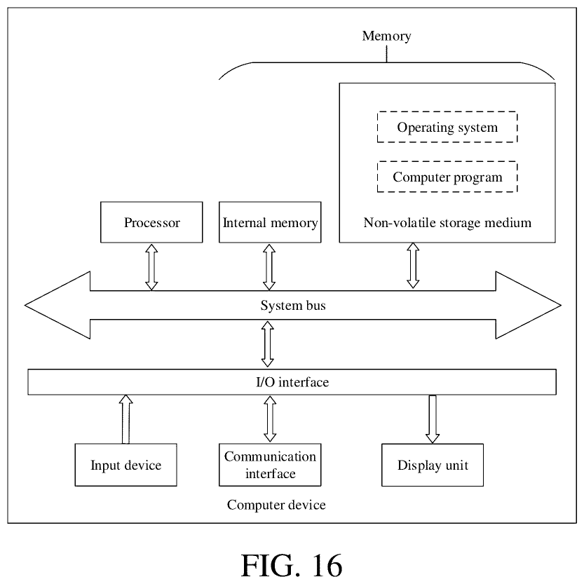

In an exemplary embodiment, a computer device is provided. The computer device may be a terminal, and an internal structural diagram of the computer device may be shown in FIG. 16. The computer equipment includes a processor, a memory, an input/output interface, a communication interface, a display unit, and an input device. The processor, the memory, and the input/output interface are connected via a system bus, and the communications interface, the display unit, and the input apparatus are connected to the system bus via the input/output interface. The processor of the computer device is configured to provide computing and control capabilities. The memory of the computer device includes a non-volatile storage medium and an internal memory. The non-volatile storage medium stores an operating system and a computer program. The internal memory provides an environment for the operation of the operating system and the computer program in the non-volatile storage medium. The input/output interface of the computer device is configured to exchange information between the processor and an external device. The communications interface of the computer device is configured to perform wired or wireless communication with an external terminal, and the wireless communication may be implemented by using WIFI, a mobile cellular network, near field communication (NFC), or other technologies. The computer program is executed by the processor to implement a method for obtaining a contrast scanning protocol. The display unit of the computer device is configured to form a visually perceptible images, and may be a display, a projection apparatus, or a virtual reality imaging apparatus. The display may be a liquid crystal display or an electronic ink display. The input device of the computer device may be a touch layer covering on the display, or may be a key, a trackball, or a touchpad disposed on a housing of the computer device, or may be external devices such as a keyboard, touchpad, or mouse.

Those skilled in the art may understand that the structures shown in FIG. 15 and FIG. 16 are merely block diagrams of partial structures related to the solutions of the present disclosure, and do not constitute a limitation on a computer device to which the solution of the present disclosure is applied. A specific computer device may include more or fewer components than those shown, or combine some components, or have different component arrangements.

In an embodiment, a computer device is provided, including a memory and a processor. The memory stores a computer program that, when executed by the processor, implements the steps in the method embodiments described above.

In an embodiment, a computer-readable storage medium is provided, on which a computer program is stored. The computer program, when executed by a processor, implements the steps in the method embodiments described above.

In an embodiment, a computer program product is provided, including a computer program that, when executed by the processor, implements the steps in the method embodiments described above.

It should be noted that user information (including but not limited to user device information, personal user information, and the like) and data (including but not limited to data used for analysis, stored data, displayed data, and the like) involved in the present disclosure are information and data authorized by users or fully authorized by all relevant parties. The collection, use, and processing of related data need to comply with relevant regulations.

Those skilled in the art can understand that all or part of the processes in the above method embodiments may be implemented by a computer program to instruct related hardware, and the computer program may be stored in a non-volatile computer-readable storage medium. The computer program, when executed, may implement the process in the embodiments of the methods described above. Any reference to a memory, a database, or other media used in the embodiments provided in the present disclosure may include at least one of a non-volatile memory and a volatile memory. The non-volatile memory may include read-only memory (ROM), magnetic tape, floppy disk, flash memory, optical memory, high-density embedded non-volatile memory, resistive random access memory (ReRAM), magnetic resistive random access memory (MRAM), ferroelectric random access memory (FRAM), phase change memory (PCM), graphene memory, or the like. The volatile memory may include a random access memory (RAM), an external cache memory, or the like. By way of illustration and not limitation, the RAM may be in various forms, such as a static random access memory (SRAM) or a dynamic random access memory (DRAM). The database in the embodiments provided in the present disclosure may include at least one of a relational database and a non-relational database. The non-relational database may include a blockchain-based distributed database and the like, and is not limited thereto. The processor in the embodiments provided in the present disclosure may be a general-purpose processor, a central processing unit, a graphics processing unit, a digital signal processor, a programmable logic device, a quantum computing-based data processing logic, an artificial intelligence (AI) processor, or the like, and is not limited thereto.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above embodiments only illustrate several implementations of the present disclosure, and the description thereof is specific and detailed, but cannot therefore be understood as limiting the protection scope of the present disclosure. It should be noted that those of ordinary skill in the art may further make variations and improvements without departing from the conception of the present disclosure, and these all fall within the protection scope of the present disclosure. Therefore, the protection scope of the present disclosure should be subject to the appended claims.

What is claimed is:

1. A method of determining a contrast scanning protocol, comprising:
obtaining a target scanned object physiological model of a target scanned object, and inputting the target scanned object physiological model and a plurality of candidate contrast scanning protocols into a trained contrast simulation model to obtain enhancement effect parameters of the plurality of candidate contrast scanning protocols; and
determining a recommended contrast scanning protocol from the plurality of candidate contrast scanning protocols according to the enhancement effect parameters of the plurality of candidate contrast scanning protocols.

2. The method of claim 1, wherein obtaining the target scanned object physiological model of the target scanned object further comprises:
obtaining target scanned region information and target injection region information of the target scanned object, and obtaining a general physiological model according to the target scanned region information and the target injection region information, wherein the general physiological model characterizes physiological structural information between the target scanned region and the target injection region; and
adjusting, based on vital signs information of the target scanned object, the general physiological model to obtain the target scanned object physiological model corresponding to the target scanned object.

3. The method of claim 2, wherein adjusting, based on the vital signs information of the target scanned object, the general physiological model to obtain the target scanned object physiological model corresponding to the target scanned object further comprises:
determining the vital signs information by performing vital signs information extraction on the target scanned object, wherein the vital signs information comprises either or both of physiological parameters affecting changes in fluid flow characteristics in the target scanned region and medical information of the target scanned object; and
adjusting the general physiological model according to the extracted vital signs information to obtain the target scanned object physiological model corresponding to the target scanned object.

4. The method of claim 1, wherein the plurality of candidate contrast scanning protocols are acquired by:
determining a setting range of contrast injection parameters and a setting range of contrast scanning parameters;
determining a plurality of injection parameters in the setting range of contrast injection parameters and a plurality of scanning parameters in the setting range of contrast scanning parameters; and
obtaining the plurality of candidate contrast scanning protocols according to a combination result of the plurality of injection parameters and the plurality of scanning parameters.

5. The method of claim 1, wherein determining the recommended contrast scanning protocol from the plurality of candidate contrast scanning protocols according to the enhancement effect parameters of the plurality of candidate contrast scanning protocols further comprises:
obtaining a preset target enhancement effect parameter, and determining similarities between the enhancement effect parameters of the candidate contrast scanning protocols and the preset target enhancement effect parameter; and
designating a candidate contrast scanning protocol whose similarity satisfies a preset similarity condition as the recommended contrast scanning protocol.

6. The method of claim 5, wherein obtaining the preset target enhancement effect parameter further comprises:
determining the target enhancement effect parameter according to at least one of an imaging enhancement value, image quality evaluation information, or an image signal-to-noise ratio corresponding to a target medical image, wherein the target medical image is a medical image that is expected to be obtained after performing contrast scanning based on a candidate contrast scanning protocol corresponding to the target medical image.

7. The method of claim 1, wherein the contrast simulation model is configured to simulate one or more of flow characteristics of a contrast agent, the one or more flow characteristics comprising:
a time-varying distribution process of the contrast agent, a time for the contrast agent to reach the target scanned region, or a time for the contrast agent to exit the target scanned region.

8. The method of claim 1, wherein a training process of the contrast simulation model comprises:
determining a sample contrast scanning protocol configured for a sample scanned object to acquire an expected image enhancement effect, and determining, according to an actual enhancement effect obtained when performing sample contrast scanning on the sample scanned object based on the sample contrast scanning protocol, first enhancement effect evaluation information that is taken as a label;
inputting sample vital signs information of the sample scanned object, a sample enhancement effect parameter corresponding to the expected image enhancement effect, and the sample contrast scanning protocol into an original simulation model to obtain second enhancement effect evaluation information output by the original simulation model; and
adjusting the original simulation model according to a difference between the second enhancement effect evaluation information and the first enhancement effect evaluation information until a training end condition is satisfied, to obtain the trained contrast simulation model.

9. The method of claim 8, wherein inputting the sample vital signs information of the sample scanned object, the sample enhancement effect parameter corresponding to the expected image enhancement effect, and the sample contrast scanning protocol into the original simulation model to obtain the second enhancement effect evaluation information output by the original simulation model, further comprises:
inputting the sample vital signs information of the sample scanned object, the sample enhancement effect parameter corresponding to the expected image enhancement effect, and the sample contrast scanning protocol into the original simulation model; and
obtaining, by the original simulation model, sample vital signs information vectors corresponding to the sample scanned object, a sample enhancement effect vector corresponding to the sample enhancement effect parameter, and a contrast scanning protocol vector corresponding to the sample contrast scanning protocol, determining, by the original simulation model, a feature combination result according to the sample vital signs information vectors, and outputting, by the original simulation model, the second enhancement effect evaluation information according to the feature combination result, a sample enhancement effect feature corresponding to the sample enhancement effect vector, and a contrast scanning protocol feature corresponding to the contrast scanning protocol vector.

10. The method of claim 9, wherein the original simulation model comprises an input layer, a processing layer, a hidden layer, and an output layer that are sequentially connected;
obtaining, by the original simulation model, sample vital signs information vectors corresponding to the sample scanned object, the sample enhancement effect vector corresponding to the sample enhancement effect parameter, and the contrast scanning protocol vector corresponding to the sample contrast scanning protocol, further comprises:
obtaining, by the input layer, sample vital signs information vectors corresponding to the sample scanned object, the sample enhancement effect vector corresponding to the sample enhancement effect parameter, and the contrast scanning protocol vector corresponding to the sample contrast scanning protocol; and
determining, by the original simulation model, the feature combination result according to the sample vital signs information vectors, and outputting, by the original simulation model, the second enhancement effect evaluation information according to the feature combination result, the sample enhancement effect feature corresponding to the sample enhancement effect vector, and the contrast scanning protocol feature corresponding to the contrast scanning protocol vector, further comprises:
inputting the sample vital signs information vectors, the sample enhancement effect vector, and the contrast scanning protocol vector into the processing layer for feature extraction, and obtaining the feature combination result corresponding to the sample vital signs information vectors, the sample enhancement effect feature corresponding to the sample enhancement effect vector, and the contrast scanning protocol feature corresponding to the contrast scanning protocol vector; and
inputting the feature combination result, the sample enhancement effect feature, and the contrast scanning protocol feature into the hidden layer for feature fusion, inputting a feature fusion result into the output layer, and obtaining the second enhancement effect evaluation information output by the output layer.

11. The method of claim 9, wherein the feature combination result comprises features acquired in at least one of the following manners:
determining consecutive features according to sample vital signs information vectors of first-type sample vital signs information in a plurality of pieces of sample vital signs information;
encoding sample vital signs information vectors of second-type sample vital signs information in the plurality of pieces of sample vital signs information to obtain a discrete feature according to an encoding result;
performing cross-combination on sample vital signs information vectors of third-type sample vital signs information in the plurality of pieces of sample vital signs information to obtain a cross-combination feature according to a cross-combination result; or
performing feature extraction on sample vital signs information vectors of fourth-type sample vital signs information in the plurality of pieces of sample vital signs information to obtain a deep feature.

12. The method of claim 1, further comprising:
obtaining one or more optimized contrast scanning protocols according to protocol adjustment information entered by a user for the recommended contrast scanning protocol; and
adjusting the trained contrast simulation model for updating according to differences between the one or more optimized contrast scanning protocols and the recommended contrast scanning protocol, to obtain an updated trained contrast simulation model that matches a protocol configuration habit of the user.

13. The method of claim 12, wherein each of the one or more optimized contrast scanning protocols is corresponding to a contrast purpose, and the contrast purpose indicates a medical task applied to a medical image obtained after performing the contrast scanning based on the optimized contrast scanning protocol; and
adjusting the trained contrast simulation model for updating according to differences between the one or more optimized contrast scanning protocols and the recommended contrast scanning protocol, to obtain the updated trained contrast simulation model that matches the protocol configuration habit of the user, further comprises:
determining a plurality of optimized contrast scanning protocols corresponding to the same contrast purpose; and
for each contrast purpose, adjusting the trained contrast simulation model according to differences between the plurality of optimized contrast scanning protocols corresponding to the contrast purpose and a corresponding recommended contrast scanning protocol, to obtain the updated trained contrast simulation model that matches the protocol configuration habit of the user and the contrast purpose.

14. A method of determining a contrast scanning protocol, comprising:
obtaining a candidate contrast scanning protocol of a target scanned object, a target enhancement effect parameter configured to indicate an expected image enhancement effect for a user, and vital signs information of the target scanned object;
inputting the candidate contrast scanning protocol, the target enhancement effect parameter, and the vital signs information of the target scanned object into a trained contrast simulation model, to obtain enhancement effect evaluation information of the candidate contrast scanning protocol that is output by the trained contrast simulation model, wherein the enhancement effect evaluation information indicates a degree of difference between an image enhancement effect obtained based on the candidate contrast scanning protocol and the expected image enhancement effect; and
determining a recommended contrast scanning protocol according to the enhancement effect evaluation information.

15. The method of claim 14, wherein a training process of the contrast simulation model comprises:
determining a sample contrast scanning protocol configured for a sample scanned object to acquire an expected image enhancement effect, and determining, according to an actual enhancement effect obtained when performing sample contrast scanning on the sample scanned object based on the sample contrast scanning protocol, first enhancement effect evaluation information that is taken as a label; and
inputting sample vital signs information of the sample scanned object, a sample enhancement effect parameter corresponding to the expected image enhancement effect, and the sample contrast scanning protocol into an original simulation model to obtain second enhancement effect evaluation information output by the original simulation model; and
adjusting the original simulation model according to a difference between the second enhancement effect evaluation information and the first enhancement effect evaluation information until a training end condition is satisfied, to obtain a trained contrast simulation model.

16. The method of claim 15, wherein inputting the sample vital signs information of the sample scanned object, the sample enhancement effect parameter corresponding to the expected image enhancement effect, and the sample contrast scanning protocol into the original simulation model to obtain the second enhancement effect evaluation information output by the original simulation model, further comprises:
inputting the sample vital signs information of the sample scanned object, the sample enhancement effect parameter corresponding to the expected image enhancement effect, and the sample contrast scanning protocol into the original simulation model;
obtaining, by the original simulation model, sample vital signs information vectors corresponding to the sample scanned object, a sample enhancement effect vector corresponding to the sample enhancement effect parameter, and a contrast scanning protocol vector corresponding to the sample contrast scanning protocol, determining, by the original simulation model, a feature combination result according to the sample vital signs information vectors, and outputting, by the original simulation model, the second enhancement effect evaluation information according to the feature combination result, a sample enhancement effect feature corresponding to the sample enhancement effect vector, and a contrast scanning protocol feature corresponding to the contrast scanning protocol vector.

17. The method of claim 16, wherein the original simulation model comprises an input layer, a processing layer, a hidden layer, and an output layer that are sequentially connected;
obtaining, by the original simulation model, sample vital signs information vectors corresponding to the sample scanned object, the sample enhancement effect vector corresponding to the sample enhancement effect parameter, and the contrast scanning protocol vector corresponding to the sample contrast scanning protocol, further comprises:
obtaining, by the input layer, sample vital signs information vectors corresponding to the sample scanned object, the sample enhancement effect vector corresponding to the sample enhancement effect parameter, and the contrast scanning protocol vector corresponding to the sample contrast scanning protocol; and
determining, by the original simulation model, the feature combination result according to the sample vital signs information vectors, and outputting, by the original simulation model, the second enhancement effect evaluation information according to the feature combination result, the sample enhancement effect feature corresponding to the sample enhancement effect vector, and the contrast scanning protocol feature corresponding to the contrast scanning protocol vector, further comprises:
inputting the sample vital signs information vectors, the sample enhancement effect vector, and the contrast scanning protocol vector into the processing layer for feature extraction, and obtaining the feature combination result corresponding to the sample vital signs information vectors, the sample enhancement effect feature corresponding to the sample enhancement effect vector, and the contrast scanning protocol feature corresponding to the contrast scanning protocol vector; and
inputting the feature combination result, the sample enhancement effect feature, and the contrast scanning protocol feature into the hidden layer for feature fusion, inputting a feature fusion result into the output layer, and obtaining the second enhancement effect evaluation information output by the output layer.

18. The method of claim 17, wherein the feature combination result comprises features acquired in at least one of the following manners:
determining consecutive features according to sample vital signs information vectors of first-type sample vital signs information in a plurality of pieces of sample vital signs information; and
encoding sample vital signs information vectors of second-type sample vital signs information in the plurality of pieces of sample vital signs information to obtain a discrete feature according to an encoding result;
performing cross-combination on sample vital signs information vectors of third-type sample vital signs information in the plurality of pieces of sample vital signs information to obtain a cross-combination feature according to a cross-combination result; or
performing feature extraction on sample vital signs information vectors of fourth-type sample vital signs information in the plurality of pieces of sample vital signs information to obtain a deep feature.

19. The method of claim 14, further comprising:
obtaining one or more optimized contrast scanning protocols according to protocol adjustment information entered by a user for the recommended contrast scanning protocol; and
adjusting the trained contrast simulation model for updating according to differences between the one or more optimized contrast scanning protocols and the recommended contrast scanning protocol, to obtain an updated trained contrast simulation model that matches a protocol configuration habit of the user.

20. A computer device, comprising a memory and a processor, wherein the memory stores a computer program, and when the computer program is executed by the processor, the method of claim 1 is realized.
